(19) United States
(12) Patent Application Publication
(10) Pub. No.: US 2012/0288162 A1
(43) Pub. Date: Nov. 15, 2012

(54) METHOD OF INCREASING SOIL RESOURCE CAPTURE IN A PLANT

(75) Inventor: Jonathan P. Lynch, Boalsburg, PA (US)

(73) Assignee: THE PENN STATE RESEARCH FOUNDATION, University Park, PA (US)

(21) Appl. No.: 13/574,625

(22) PCT Filed: Jan. 26, 2011
(86) PCT No.: PCT/US11/22596

§ 371 Date: Jul. 23, 2012

Related U.S. Application Data
(60) Provisional application No. 61/298,424, filed on Jan. 26, 2010.
(60) Provisional application No. 61/353,513, filed on Jun. 10, 2010.

(51) Int. Cl. G06K 9/00; A01H 1/04; A01C 11/00; A01H 1/02

(52) U.S. Cl. 382/110; 800/260; 800/298; 111/100

(57) ABSTRACT

The invention is directed to improving a plants ability to acquire soil resources. Plants that develop a greater amount of aerenchyma will have an improved ability to acquire soil resources and will have greater drought tolerances. Such plants will grow faster and larger than plants that have less aerenchyma. Farmers who plant crops with seeds that will grow into plants have more aerenchyma will be able to reduce the amount of fertilizer used in growing a crop. Accordingly, the invention relates to identifying and breeding a plant with a greater amount of aerenchyma. The identified plant and its progeny will have an improved ability to capture soil resources and will be tolerant to drought.

### CROSS REFERENCE TO RELATED APPLICATIONS

This application claims the benefit of U.S. provisional application No. 61/298,424 titled “Root cortical aerenchyma as a selection trait for drought tolerance in plants,” filed on Jan. 26, 2010; and U.S. provisional application No. 61/353,513 titled “Root cortical aerenchyma as a selection trait for abiotic stress tolerance in plants,” filed on Jun. 10, 2010. Each provisional application identified above is incorporated herein by reference.

### STATEMENT REGARDING FEDERALLY SPONSORED RESEARCH

This invention was made in part with United States Government support awarded by the United States Department of Agriculture under grant number NRI 2007-35100-18365; the Dry Grain Pulses Collaborative Research Program of the United States AID-Development Grant program under grant number EDH-A-00-07-00005-00, the National Science Foundation and the National Science Foundation/BREAD under grant number 0965380. Therefore, the United States Government may have certain rights in this invention.

### BACKGROUND OF THE INVENTION

1. Field of the Invention

This invention relates generally to the field of identifying, selecting and creating plants with improved ability to capture soil resources or improved drought tolerance. Such plants can be grown under low soil resource conditions, or will have improved growth when grown with adequate soil resources, as compared to plants lacking this improved ability to capture soil resources.

2. Description of Related Art

Root cortical aerenchyma (henceforth “aerenchyma”) is an air-filled space in the root cortex. It is formed by programmed cell death, which creates large intercellular spaces in the cortical root tissue. This promotes gas exchange between the shoots and roots. For example, it enhances the diffusion of atmospheric or photosynthetic oxygen from the shoot to the root and rhizosphere. It is known to develop in plants that have been exposed to flooding. The gas exchange promoted by aerenchyma allows for the plant to tolerate hypoxic conditions during flooding.

In order for a plant to grow, it must access soil resources such as water or soil nutrients. Drought or soil with nutrient deficiencies can cause abiotic stress resulting in reduced plant growth. For example, a nitrogen-deficient plant is generally small and develops slowly because it lacks the nitrogen necessary for optimal photosynthesis, growth and metabolism. The same is true of water, phosphorus, potassium, and other essential resources.

Aerenchyma formation is stimulated in response to hypoxia. Under well-drained conditions, aerenchyma was previously believed to cause limitations to root function (Skinner et al. “Aerenchyma development in native warm-season grass cultivars,” Proceedings of the Third Eastern Native Grass Symposium (2004): 48-52). This limitation was believed to cause a reduction in a plant's ability to capture soil resources, such as water and soil minerals (Benz et al. “Aerenchyma development and elevated alcohol dehydrogenase activity as alternative responses to hypoxic soils in the Piriqueta caroliniana complex,” AM. J. OF BOTANY (2007) 94(4): 542-550). Thus, aerenchyma has not been considered a phenotypic trait that is likely to benefit a plant unless the plant is stressed by flooding as a means for the plant to survive hypoxia.

### SUMMARY OF THE INVENTION

However, the inventor has surprisingly discovered that plants having a greater amount of aerenchyma have an increased ability to capture soil resources. In specific embodiments, the soil resource is water or nitrogen. Thus, plants that develop a greater amount of aerenchyma will grow faster and larger than plants that have less aerenchyma. This also provides a farmer the ability to reduce the amount of fertilizer used in growing a crop. Accordingly, the invention relates to identifying a plant with a greater amount of aerenchyma.

In one embodiment, the invention is directed to a method of increasing capture of a soil resource by a plant. The method comprises determining an amount of root cortical aerenchyma in a first plant, and an amount of root cortical aerenchyma in a second plant. The amounts of root cortical aerenchyma present in each plant are compared. An improved soil resource capturing plant is identified by comparing the amounts of root cortical aerenchyma in each plant. If the first plant has a greater amount of root cortical aerenchyma compared to a second plant, then the first plant is the improved soil resource capturing plant. If the second plant has a greater amount of root cortical aerenchyma, then the second plant is the improved soil resource capturing plant.

Once the improved soil resource capturing plant is identified, the plant can be used to produce seeds or other progeny. Seeds can be produced by sexually reproducing or asexually reproducing the improved soil resource capturing plant, thereby forming progeny of the improved soil resource capturing plant.

In another embodiment, the invention is directed to a method of reducing an amount of fertilizer used on a crop. The method comprises determining a first amount of root cortical aerenchyma in a first plant, and determining a second amount of root cortical aerenchyma in a second plant. An improved soil resource capturing plant is identified by comparing the first amount of root cortical aerenchyma in the first plant and the second amount of root cortical aerenchyma in the second plant. If the first plant has a greater amount of root cortical aerenchyma compared to a second plant, then the first plant is the improved soil resource capturing plant. If the second plant has a greater amount of root cortical aerenchyma, then the second plant is the improved soil resource capturing plant. Seeds comprising the root cortical aerenchyma phenotype observed in the improved soil resource capturing plant are planted, thereby reducing the amount of fertilizer required by the crops because the plant has an increased ability to capture soil resources. The seeds can be generated by sexually reproducing or asexually reproducing the improved soil resource capturing plant.

In another embodiment, the invention is directed to a method for determining a plant having an improved ability to capture a soil resource. The method comprises determining a first amount of root cortical aerenchyma in a first plant, and determining a second amount of root cortical aerenchyma in a second plant. An improved soil resource capturing plant is identified by comparing the first amount of root cortical aerenchyma in the first plant and the second amount of root cortical aerenchyma in the second plant. If the first plant has a greater amount of root cortical aerenchyma compared to a second plant, then the first plant is the improved soil resource capturing plant. If the second plant has a greater amount of root cortical aerenchyma, then the second plant is the improved soil resource capturing plant.

The inventor has also unexpectedly discovered that a greater amount of aerenchyma in a plant provides drought resistance to that plant. Thus, in one embodiment, the invention is directed to a method of increasing drought resistance of a plant. The method comprises determining a first amount of root cortical aerenchyma in a first plant, and a second amount of root cortical aerenchyma in a second plant. The amounts of root cortical aerenchyma present in each plant are compared. A drought resistant plant is identified by comparing the first amount of root cortical aerenchyma in the first plant and the second amount of root cortical aerenchyma in the second plant. If the first plant has a greater amount of root cortical aerenchyma compared to a second plant, then the first plant is the improved soil resource capturing plant. If the second plant has a greater amount of root cortical aerenchyma, then the second plant is the improved soil resource capturing plant.

Once the drought resistant plant is identified, the plant can be used to produce seeds or progeny. Seeds can be produced by sexually reproducing or asexually reproducing the improved soil resource capturing plant, thereby forming a progeny of the improved soil resource capturing plant.

In another embodiment, the invention is directed to a seed for a plant having an improved ability to capture a soil resource, or a drought tolerant plant. The seed is prepared by determining a first amount of root cortical aerenchyma in a first plant, and a second amount of root cortical aerenchyma in a second plant. The amounts of root cortical aerenchyma present in each plant are compared. A parent plant is identified by comparing the first amount of root cortical aerenchyma in the first plant and the second amount of root cortical aerenchyma in the second plant. If the first plant has a greater amount of root cortical aerenchyma compared to a second plant, then the first plant is the parent plant. If the second plant has a greater amount of root cortical aerenchyma, then the second plant is the parent plant. Once the parent plant is identified, the parent plant is used to produce a seed. The seed can be produced by sexually reproducing or asexually reproducing the parent plant, thereby forming the seed for the plant having the improved soil resource capturing plant, or the drought tolerant plant. The seed can optionally be planted, thereby forming the plant having the improved ability to capture soil resource, or the drought tolerant plant.

In another embodiment, the invention is directed to a plant breeding program. The breeding program generates plants that have an improved ability to capture a soil resource or a drought tolerant plant. The method comprises determining a first amount of root cortical aerenchyma in a first plant, and a second amount of root cortical aerenchyma in a second plant. The amounts of root cortical aerenchyma present in each plant are compared. A parent plant is identified by comparing the first amount of root cortical aerenchyma in the first plant and the second amount of root cortical aerenchyma in the second plant. If the first plant has a greater amount of root cortical aerenchyma compared to a second plant, then the first plant is the parent plant. If the second plant has a greater amount of root cortical aerenchyma, then the second plant is the parent plant. Once the parent plant is identified, the parent plant is used to produce a seed. The seed can be produced by sexually reproducing or asexually reproducing the parent plant, thereby forming a seed for the plant having the improved soil resource capturing plant, or the drought tolerant plant. The parent plant or a plant grown from the seed are sexually or asexually bred, wherein the breeding program ensures that progeny developed in the breeding program maintains the greater amount of root cortical aerenchyma.

### BRIEF DESCRIPTION OF THE DRAWINGS

FIG. 1 is a bar graph showing the production of root cortical aerenchyma of six genotypes at 35 days after planting in both well-watered (WW) and water-stressed (WS) conditions in the mesocosms. The data shown are means±SE of the mean (n=4). Means with the same letters are not significantly different (P≦0.05).

FIG. 2 is a bar graph showing shoot dry weight of six genotypes at 35 days after planting in both well-watered (WW) and water-stressed (WS) conditions in the mesocosms. Data shown are means±SE of the mean (n=4). Means with the same letters are not significantly different (P≦0.05).

FIG. 3 is a graph showing specific seminal root respiration per length (upper panel) or weight (lower panel) of six genotypes at 35 days after planting (DAP) in both well-watered (WW) and water-stressed (WS) conditions in the mesocosms. Data shown are means±SE of the mean (n=4). Means with the same letters are not significantly different (P≦0.05).

FIG. 4 is a graph showing root length distribution in soil layer of six genotypes (low root cortical aerenchyma (RCA) lines on the left, and high RCA lines on the right) at 35 DAP in both well-watered (WW) and water-stressed (WS) conditions in the mesocosms. Data shown are means±SE of the mean (n=4). ** shows significantly different paired means in WS and WW (P≦0.05).

FIG. 5 is a graph showing the correlation of root dry weight and shoot dry weight (upper panel), and root dry weight and percentage of root cortical aerenchyma (lower panel) for six maize lines at 35 DAP in the mesocosms. Covariate analysis showed root dry weight was non-significant for percentage of RCA (F=0.65, P=0.57). The data shown are means from four replicates.

FIG. 6 is a graph showing the results of field soil volumetric water content at 25 and 50 cm depths in both well-watered (WW) and water-stressed (WS) conditions throughout the growing season in 2008. The data shown are means±SE of the mean (n=4).

FIG. 7 is a graph showing the production of RCA of seven genotypes in both well-watered (WW) and water-stressed (WS) conditions in the field. The data shown are means±SE of the mean (n=4). Means with the same letters are not significantly different (P 0.05).

FIG. 8 is a graph showing root length density (upper panel) and root number density (lower panel) at 56 DAP from 40 to 50 cm deep soil core segments for seven genotypes in both well-watered (WW) and water-stressed (WS) conditions in the field. Data shown are means±SE of the mean (n=4). Means with the same letters are not significantly different (P≦0.05).

FIG. 9 is a graph showing root length per frame (upper panel) and root counts per frame (lower panel) by minirhizotron technique at 42 DAP at 50 cm deep soil for seven genotypes in both well-watered (WW) and water-stressed (WS) conditions in the field. Data shown are means±SE of the mean (n=4). Means with the same letters are not significantly different (P≦0.05).

FIG. 10 is a graph showing correlations between measurements made at 50 cm deep soil by minirhizotron technique and 40-50 cm deep by soil coring technique for root length (upper panel) and root number (lower panel) in the field.

FIG. 11 is a graph showing leaf relative water content at 56 d after planting (DAP) for seven genotypes in well-watered (WW) and water-stressed (WS) conditions in the field. Data shown are means±SE of the mean (n=4). Means with the same letters are not significantly different (P≦0.05).

FIG. 12 is a graph showing shoot dry weight at flowering stage (upper panel) and grain yield (lower panel) of seven genotypes in well-watered (WW) and water-stressed (WS) conditions in the field. Data shown are means±SE of the mean (n=4). Means with the same letters are not significantly different (P≦0.05).

FIG. 13 is a graph showing seed number per plant and individual seed dry weight of seven genotypes at flowering stage in well-watered (WW) and water-stressed (WS conditions in the field. Data shown are means±SE of the mean (n=4). Means with the same letters are not significantly different (P≦0.05).

FIG. 14 is a table of references used for parameters in SimRoot.

FIG. 15 is a graph showing data points taken from 30 different publications, which were independent from the SimRoot model discussed herein. The lines show simulated data of non-stressed beans and maize without RCA.

FIG. 16 is a visualization of the simulated root architecture of bean and maize at 40 days after germination in high soil phosphorus (18 μM) (HP), and low soil phosphorus (3 μM) (LP).

FIG. 17 is a graph showing shoot biomass production and root length after 40 days growth at different levels of soil phosphorus. The lines show results for plants with RCA (continuous lines) and without RCA (dashed lines) formation.

FIG. 18 is a graph showing cumulative carbon expenditure for bean and maize with and without RCA formation. Carbon sources are seed reserves and photosynthesis. HP=high soil phosphorus (18 μM), LP=low soil phosphorus (3 μM).

FIG. 19 is a graph showing nutrient stress on a scale from 0-1 as it develops over time. Stress here is calculated as 1−(u−m)/(o−m), where u is the phosphorus uptake, o is the optimal phosphorus content in the plant and m is the minimal phosphorus content in the plant. “0” indicates no stress, “1” indicates severe stress. HP=high soil phosphorus (18 μM), MP=medium soil phosphorus (6 μM), LP=low soil phosphorus (3 μM).

FIG. 20 is a graph showing relative benefit of RCA over time at a soil phosphorus availability of 3 μM. Relative benefit was calculated as 100*(X_{RCA}−X_{_{—}}_{RCA})/X_{_{—}}_{RCA}. 100 indicates 100% increase in plant dry weight due to the formation of RCA. Different lines show benefit for different functions of RCA.

FIG. 21 is a graph showing relative benefit of RCA formation at different levels of soil phosphorus availability. A line shows the hypothetical benefit if the different functions were added.

FIG. 22 is a graph showing sensitivity analysis for RCA formation. Different lines correspond with phosphorus availability in the soil, lighter lines for when phosphorus availability is lower.

FIG. 23 is a graph showing a comparison between the measured and simulated nitrate content in the different soil layers. Weekly measurements of the nitrate content of the top six, 10 cm thick, soil layers were taken in a fertilized and a non-fertilized field on the Russell E. Larson Experimental Farm at Rock Springs, Pa. The soil type was a Hager Town silt loam on which the OHW population was grown. The simulation results are from two simulations of a fertilized and non-fertilized soil in which growth of the inbred line H99 was simulated, which is one of the parents of the recombinant inbred line.

FIG. 24 is an image showing a simulation of root density and a hypothetical neighboring plant.

FIG. 25 contains graphs showing the effect of aerenchyma on plant dry weight.

FIG. 26 contains graphs showing the benefits of aerenchyma in plants with lateral roots and plants without aerenchyma in lateral roots.

FIG. 27 shows images of root architecture for H99, W64A, Pioneer 36H56 and normal plants.

FIG. 28 contains graphs showing the amount of aerenchyma developed when plants were grown under nitrate, phosphorus or potassium deficiencies. The plants that were grown under these stresses were H99, 36H56 and W64A.

FIG. 29 is a graph showing the benefit of aerenchyma to a plants growth when grown under various nutrient levels.

FIG. 30 is a graph showing nitrate leaching in silt-loam and loamy-sand soil as a function of depth.

FIG. 31 contains graphs showing the effect of aerenchyma in silt loam and loamy sand soils.

FIG. 32 is a graph showing the effect of aerenchyma in silt-loam and loamy-sand soils.

FIG. 33 is a graph showing the simulated and measured nitrate content in fertilized and unfertilized fields.

FIG. 34 is a graph showing the potassium uptake under the Barber-Cushman and SWMS-3D models.

FIG. 35 is an image showing a simulated root under the Barber-Cushman and SWMS-3D models.

FIG. 36 contains graphs showing the simulated effect of potassium availability and aerenchyma on plant dry weight.

FIG. 37 is a graph showing the nitrate uptake under the Barber-Cushman and SWMS-3D models.

### DESCRIPTION OF THE PREFERRED EMBODIMENT(S)

All numbers used in the specification and claims are to be understood as being modified in all instances by the term “about”. Accordingly, unless indicated to the contrary, the numerical values set forth in the following specification and claims may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Moreover, all ranges disclosed herein are to be understood to encompass any and all subranges subsumed therein. For example, a stated range of “1 to 10” should be considered to include any and all subranges between and inclusive on the minimum value of 1 and the maximum value of 10; that is, all subranges beginning with a minimum value of 1 or more and ending with a maximum value of 10 or less, e.g., 1.0 to 7.8, 3.0 to 4.5, and 6.3 to 10.0.

The invention relates to planting plants that will have a greater amount of aerenchyma as compared to other plants. A plant having a greater amount of aerenchyma will improve the plant's capture of a soil resource, increase the plant's tolerance to drought, and increase the plant's tolerance to low soil mineral conditions. It will also have roots that penetrate deeper into the soil. Thus, the invention is directed to a method of increasing capture of a soil resource, a method of reducing the amount of fertilizer used on a crop, a method of identifying or developing a plant having an improved ability to capture a soil resource, a method of increasing root growth of a plant, a method of increasing growth of a plant under drought conditions, and a method of increasing growth of a plant under suboptimal soil mineral conditions.

In each of these methods, a plant having a greater amount of aerenchyma is identified. The plant is identified by methods known in the art. For example, two plants can be grown in soil having similar amounts of soil resources. The soil can provide the plants with adequate resources or with suboptimal resource thereby placing the plant under stress. For example, the plants can be grown under drought conditions or with suboptimal nitrogen. Once grown for a period of days after planting, a section of the root from each plant is examined visually. The visual examination can be aided with a microscope. Through the visual inspection, one can compare the amount of aerenchyma present in each section. Optionally, images from the microscope can be obtained and thereafter analyzed visually or by a computer.

Another method to determine the amount of aerenchyma is by weighing the root to determine the amount of air space within the root. This is accomplished by pre-weighing a volume of degassed water. A section of root is added to the degassed water and pre-weighed. The section of root is moved to a vial also filled with degassed water, and then vacuum-infiltrated until no more bubbles are released from the section of root. The vacuum-infiltrated root tissue is returned to the pre-weighed water, and the water with the root tissue is re-weighed. Porosity of the root can be calculated with the following formula:

Porosity (%)=100×[(FA−FB)÷(FW+TW−FB)].

FA is the weight of the container for the water and the water with the root after vacuum-infiltration. FB is the weight of the container for the water and the water with the root section before vacuum-infiltration. FW is the weight of the container or the water and the degassed water without the root section. TW is the weight of the root section.

Once the amount of aerenchyma is identified in a plant, it can be compared to an amount of aerenchyma from a different plant to determine which plant will have an improved ability to acquire soil resources. The plant should be compared to a plant from the same genus, or the same species. For example, if one wishes to implement the inventive method with corn (Zea mays), one would first determine the amount of aerenchyma in a first corn plant. The amount of aerenchyma would be compared to an amount of aerenchyma in a second plant from the Zea genus, or, more preferably, a Z. mays (corn) plant. The amount of aerenchyma in the second plant can be determined by measuring the aerenchyma by the methods known within the art or discussed above, or by referring to a reference. The reference can contain information regarding the average amount of aerenchyma in the genus or species, or an amount of aerenchyma that will provide a plant with the increased ability to capture soil resources.

Preferably, the amount of aerenchyma in each plant is compared at related loci on the plant. For example, if the amount of aerenchyma for the first plant is measured at the basal region of the root, this amount should be compared to the amount of aerenchyma in a basal region of the second plant. Although aerenchyma can be measured at other loci (for example, the middle portion or the apical portion of the root region), it is preferred to measure aerenchyma at the basal region.

The amount of aerenchyma can be determined from a plant grown under well-watered and adequate soil minerals (for the purposes of this paragraph, such conditions will be referred to as “adequate conditions”), or under stress, such as under flooded conditions, drought conditions or low soil mineral availability. For example, when corn or common bean is the plant of interest, an adequate amount of phosphorus would be 1 mM (NH₄)₂HPO₄, whereas these plant species would be under stress if the amount of phosphorus present was 1 μM (NH₄)₂HPO₄. One of ordinary skill would recognize that adequate conditions for one plant species may not be adequate for another. Under adequate conditions as well as under stress, plants exhibit variation in the amount of aerenchyma the plant contains. Plants that have a greater amount of aerenchyma under stress also have a greater amount of aerenchyma under adequate conditions. This variation is more pronounced when plants are grown under stress.

The amount of aerenchyma a plants develops is dependent on many variables. They include the species of the plant, the growth conditions, and the length of time the plant is permitted to grow before determining the aerenchyma. Therefore, when comparing plants, the two plants should be grown for the same time and under similar conditions. For example, corn grown for 12 days under 1 mM phosphorus (adequate conditions) and 1 μM phosphorus (stress); and common bean grown for six weeks under similar adequate and stress conditions may have the following ranges of aerenchyma:

| | Growing | Percentage of aerenchyma as |
|---|---|---|
| Plant | conditions | basal root region |
| Corn with high aerenchyma | Adequate | 3.0 to 8.0% |
| Corn with low aerenchyma | Adequate | <3.0% |
| Corn with high aerenchyma | Stressed | 30 to 50% |
| Corn with low aerenchyma | Stressed | <30% |
| Common bean with high | Adequate | 6.0 to 15% |
| aerenchyma | | |
| Common bean with low | Adequate | <6% |
| aerenchyma | | |
| Common bean with high | Stressed | 20 to 40% |
| aerenchyma | | |
| Common bean with low | Stressed | <20% |
| aerenchyma | | |

The maximum amount of root cortical aerenchyma for a plant measured in the cortex would be approximately 80%.

The inventor has surprisingly found that a plant having more aerenchyma will have a greater ability to capture soil resources. Thus, in practicing the invention, when one plant is compared to a second plant, the plant having more aerenchyma will be the plant that has an improved ability to capture a soil resource. If the first plant and the second plant are planted under the same soil conditions and grown for the same number of days, the inventor has unexpectedly discovered that the plant having a greater amount of aerenchyma will be the plant that captures more soil resources. Consequently, this plant will grow larger than the other plant. This is true if the plant is grown under stress or under adequate conditions.

A soil resource is any resource required by the plant to grow that is found in the soil. This includes water and soil minerals. Soil minerals include nitrogen, phosphorus, potassium, calcium, magnesium, sulfur, boron, iron, chlorine, manganese, copper, silicon, molybdenum, nickel, and zinc. Within the art, soil minerals are classified into primary macronutrients, secondary macronutrients, and micronutrients. Primary macronutrients include nitrogen, phosphorus and potassium. Secondary macronutrients refer to calcium, magnesium and sulfur. The term micronutrient refers to boron, copper, iron, chlorine, manganese, molybdenum, nickel, silicon (for some plants) and zinc.

A soil resource can be mobile or immobile. Immobile resources include phosphorus and potassium whereas mobile resources include water and nitrogen. Mobile resources operate under a transportation driven mass flow dynamic. Certain embodiments of the invention are directed to improving the capture of a mobile resource, such as water or nitrogen. The inventor discovered that aerenchyma surprisingly increases a plant's ability to capture mobile resources.

One of ordinary skill in the art would recognize optimal soil resource conditions for a particular plant. Listings of such resources are readily available in publicly available references such as PLANT ANALYSIS: AN INTERPRETATION MANUEL by Robinson and Reuter, which is incorporated by reference.

If a plant has an improved ability to capture soil resources, the plant is more efficient at capturing the soil resources. Consequently, when at least a portion of a crop contains a plant having an improved ability to capture a soil resource, the amount of fertilizer a farmer will have to use is reduced because the improved soil resource capturing plant will more efficiently capture nutrients. This can result in a significant saving to the farmer in fertilizer cost, a significant increase in crop yield, and/or a significant shortening of the time between planting a plant and harvesting the crop.

Once the plant having the improved ability to capture a soil resource is identified, the plant can be grown, or it can be bred to produce other plants or seeds having this phenotype. The plant can be bred sexually or asexually by means known within the art. Examples of sexual means to breed a plant include self-pollination and cross-pollination. Examples of asexual means to reproduce the plant include budding, tillering, and apomixis. One of ordinary skill within the art would appreciate that self-pollinated or asexually reproducing the plant having the improved ability to capture a soil resource will produce a copy of the plant. Therefore, there is a greater likelihood that the self-pollinated or asexually reproduced plant would have the improved ability to capture soil resource phenotype.

Reproducing a plant, whether by sexual or asexual means, produces a progeny. The progeny can be in the form of a seed produced through the sexual or asexual reproductive process, or a plant produced through certain forms of asexual production, such as tillering. Through plant breeding, one of ordinary skill would be able to preserve the improved ability to capture soil resource phenotype in the progeny and subsequent generations.

The plants of the invention may be used in a plant breeding program. Any of a number of standard breeding techniques can be used for breeding according to the selection trait of the invention, depending upon the species to be crossed. The goal of plant breeding is to combine, in a single variety or hybrid, various desirable traits, wherein the improved ability to capture soil resources is at least one of the desired traits. This invention encompasses methods for identifying a plant having a greater amount of aerenchyma, reproducing that plant and/or producing a new plant by crossing a first parent plant with a second parent plant wherein one or both of the parent plants is a plant displaying a greater amount of aerenchyma phenotype.

For example, an amount of aerenchyma can be determined for a first plant and a second plant. Assuming that the first plant has the greater amount of aerenchyma, it will be considered the first parent plant. The second parent plant can be the second plant or a third plant. The third plant can be a progeny from the first parent plant or some other plant.

Plant breeding techniques known in the art and used in a plant breeding program include, but are not limited to, recurrent selection, bulk selection, mass selection, backcrossing, pedigree breeding, open pollination breeding, restriction fragment length polymorphism enhanced selection, genetic marker enhanced selection, doubled haploids, and transformation. Often, combinations of these techniques are used.

The development of hybrids in a plant breeding program requires, in general, the development of homozygous inbred lines, the crossing of these lines, and the evaluation of the crosses. There are many analytical methods available to evaluate the result of a cross. The oldest and most traditional method of analysis is the observation of phenotypic traits. Alternatively, the genotype of a plant can be examined.

A genetic trait which has been identified, selected or engineered into a particular plant using breeding or transformation techniques can be moved into another line using traditional breeding techniques that are well known in the plant breeding arts. For example, a backcrossing approach is commonly used to move a trait from a one maize plant to an elite inbred line, and the resulting progeny would then comprise the trait(s). Also, if an inbred line was used for trait selection, then the plants could be crossed to a different inbred line in order to produce a hybrid maize plant. As used herein, “crossing” can refer to a simple X by Y cross, or the process of backcrossing, depending on the context.

The development of a hybrid in a plant breeding program involves three steps: (1) the selection of plants from various germplasm pools for initial breeding crosses; (2) the self-crossing of the selected plants from the breeding crosses for several generations to produce a series of inbred lines, which, while different from each other, breed true and are highly homozygous; and (3) crossing the selected inbred lines with different inbred lines to produce the hybrids. During the inbreeding process, the vigor of the lines decreases. Vigor is restored when two different inbred lines are crossed to produce the hybrid. An important consequence of the homozygosity and homogeneity of the inbred lines is that the hybrid created by crossing a defined pair of inbreds will always be the same. Once the inbreds that give a superior hybrid have been identified, the hybrid seed can be reproduced indefinitely as long as the homogeneity of the inbred parents is maintained.

The above described method is applicable to any plant that can develop aerenchyma. Examples of such plants include plants from the Compositae (also known as Asteraceae), Fagaceae, Leguminosae, Poaceae (also known as Gramineae) and Rosaciae families. Specific examples of plants from the Compositae family include dahlias, lettuces, mums, sunflowers and zinnias. Specific examples of plants from the Leguminosae family include: alfalfa, beans, chickpea, clover, cowpeas, lentils, lotus, lupins, peanuts, peas, soybeans, sweet clover, sweetpea, velvet beans, vetch, wisteria and yam bean. Specific examples of plants from the Poaceae family include: barley, corn, lawn grass, millet, oats, rice, rye, sorghum, sugar cane, triticale, and wheat. Specific examples of plants from the Rosaciae family include almonds, apples, apricots, blackberries, boysenberries, cherries, loganberries, loquats, medlars, peaches, pears, pecans, plums, quinces, raspberries, roses, sloes, strawberries, and walnuts. Other examples of plants include broccoli, cabbage, canola, conifers, cotton, deciduous trees, evergreens, forest trees (e.g.: Pinus, Quercus, Pseudotsuga, Sequoia, Populus, etc.), geraniums, impatiens, nut plants (e.g. hazels, hickories, pecan, walnuts, etc.), pepper, potato, tobacco and tomato.

Examples of genera of plants that develop aerenchyma are: Acamptoclados, Achnatherum, Achnella, Acroceras, Aegilops, Aegopgon, Agroelymus, Agrohordeum, Agropogon, Agropyron, Agrositanion, Agrostis, Aira, Allium, Allolepis, Alloteropsis, Alopecurus, Amblyopyrum, Ammophila, Ampelodesmos, Amphibromus, Amphicarpum, Amphilophis, Anastrophus, Anatherum, Andropogron, Anemathele, Aneurolepidium, Anisantha, Anthaenantia, Anthephora, Anthochloa, Anthoxanthum, Antirrhinum, Apera, Apluda, Arabidopsis, Arachis, Archtagrostis, Arctophila, Argillochloa, Argyranthemum, Aristida, Arrhenatherum, Arthraxon, Arthrostylidium, Arundinaria, Arundinella, Arundo, Asparagus, Aspris, Atheropogon, Atropa, Avena, Avenella, Avenochloa, Avenula, Axonopus, Bambusa, Beckmannia, Blepharidachne, Blepharoneuron, Bothriochloa, Bouteloua, Brachiaria, Brachyelytrum, Brachypodium, Brassica, Briza, Brizopyrum, Bromelica, Bromopsis, Bromus, Browaalia, Buchloe, Bulbilis, Calamagrostis, Calamovilfa, Campulosus, Capriola, Capsicum, Catabrosa, Catapodium, Cathestecum, Cenchropsis, Cenchrus, Centaurea, Centotheca, Ceratochloa, Chaetochloa, Chasmanthium, Chimonobambusa, Chionochloa, Chloris, Chondrosum, Chrysanthemum, Chrysopon, Chusquea, Ciahorium, Cicer, Cinna, Citrus, Cladoraphis, Coelorachis, Coix, Coleanthus, Colpodium, Coridochloa, Cornucopiae, Cortaderia, Corynephorus, Cotoneaster, Cottea, Cousinia, Crataegus, Critesion, Crypsis, Ctenium, Cucumis, Cucurbita, Cutandia, Cylindropyrum, Cymbopogon, Cynara, Cynodon, Cynosurus, Cytrococcum, Dactylis, Dactyloctenium, Danthonia, Dasyochloa, Dasyprum, Datura, Dahlia, Daucus, Davyella, Dendrocalamus, Deschampsia, Desmazeria, Deyeuxia, Diarina, Diarrhena, Dichanthelium, Dichanthium, Dichelachne, Diectomus, Digitaria, Digitalis, Dimeria, Dimorpostachys, Dinebra, Diplachne, Dissanthelium, Dissochondrus, Distichlis, Drepanostachyum, Dupoa, Dupontia, Echinochloa, Ectosperma, Ehrharta, Eleusine, Elyhordeum, Elyleymus, Elymordeum, Elymus, Elyonurus, Elysitanion, Elytesion, Elytrigia, Enneapogon, Enteropogon, Epicampes, Eragrostis, Eremochloa, Eremopoa, Eremopyrum, Erianthus, Ericoma, Erichloa, Eriochrysis, Erioneuron, Euchlaena, Euclasta, Eulalia, Eulaliopsis, Eustachys, Fargesia, Festuca, Festulolium, Fingerhuthia, Fluminia, Fragaria, Garnotia, Gastridium, Gaudinia, Geranium, Gigantochloa, Glebionis, Glyceria, Glycine, Graphephorum, Gymnopogon, Gynerium, Hackelochloa, Hainardia, Hakonechloa, Haynaldia, Heleochloa, Helianthus, Helictotrichon, Hemarthria, Hesperochloa, Hesperostipa, Heterocallis, Heteropogon, Hibanobambusa, Hierochloe, Hilaria, Holcus, Homalocenchrus, Hordeum, Hydrochloa, Hymenachne, Hyoscyamus, Hyparrhenia, Hypogynium, Hystrix, Ichnanthus, Imperata, Indocalamus, Isachne, Ischaemum, Ixophorus, Juglans, Koeleria, Korycarpus, Kerria, Lactuca, Lagurus, Lamarckia, Lasiacis, Leersia, Lens, Leptochloa, Leptochloopsis, Leptocoryphium, Leptoloma, Leptogon, Lepturus, Lerchenfeldia, Leucanthemopsis, Leucanthemum, Leucopoa, Leymostachys, Leymus, Lolium, Limnodea, Linum, Lithachne, Lolium, Lophochlaena, Lophochloa, Lophopyrum, Lotus, Ludolfia, Lupinus, Luziola, Lycopersicon, Lycurus, Lygeum, Majorana, Maltea, Manihot, Manisuris, Medicago, Megastachya, Melica, Melinis, Mibora, Microchloa, Microlaena, Microstegium, Milium, Miscanthus, Mnesithea, Molinia, Monanthochloe, Monerma, Monroa, Muhlenbergia, Nardus, Nassella, Nazia, Neeragrostis, Nemesis, Neoschischkinia, Neostapfia, Neyraudia, Nicotiana, Nothoholcus, Olyra, Onobrychis, Opizia, Oplismenus, Orcuttia, Oryza Oryzopsis, Otatea, Oxytenanthera, Particularia, Panicum, Panieum, Pappophorum, Parapholis, Pascopyrum, Paspalidium, Paspalum, Pelargonium, Pennisetum, Petunia, Pisum, Phalaris, Phalaroides, Phanopyrum, Pharus, Phaseolus, Phippsia, Phleum, Pholiurus, Photinia, Phragmites, Phyllostachys, Piptatherum, Piptochaetium, Pleioblastus, Pleopogon, Pleuraphis, Pleuropogon, Poa, Podagrostis, Polypogon, Polytrias, Prunus, Psathyrostachys, Pseudelymus, Pseudoroegneria, Pseudosasa, Ptilagrostis, Puccinellia, Pucciphippsia, Pyracantha, Ranunculus, Raphanus, Redfieldia, Reimaria, Reimarochloa, Rhaphis, Rhodotypos, Rhodanthemum, Rhombolytrum, Rhynchelytrum, Roegneria, Rosa, Rostraria, Rottboellia, Rytilix, Saccharum, Sacciolepis, Salpiglossis, Sasa, Sasaella, Sasamorpha, Savastana, Schedonnardus, Schismus, Schizachne, Schizachyrium, Schizostachyum, Sclerochloa, Scleropoa, Scleropogon, Scolochloa, Scribneria, Secale, Semiarundinaria, Senecio, Sesleria, Setaria, Shibataea, Sieglingia, Sinapis, Sinarundinaria, Sinobambusa, Sinocalamus, Sitanion, Solanum, Sorbus, Sorghastrum, Sorghum, Spartina, Sphenopholis, Spiraea, Spodiopogon, Sporobolus, Stapfia, Steinchisma, Stenotaphrum, Stipa, Stipagrostis, Stiporyzopsis, Swallenia, Syntherisma, Taeniatherum, Tagetes, Tanacetum, Terrellia, Terrelymus, Thamnocalamus, Themeda, Thinopyrum, Thuarea, Thysanolaena, Torresia, Torreyochloa, Trachynia, Trachypogon, Tragus, Trichachne, Trichloris, Tricholaena, Trichoneura, Tridens, Trifolium, Triodia, Triplasis, Tripogon, Tripsacum, Trisetobromus, Trisetum, Triticosecale, Triticum, Trigonella, Tuctoria, Uniola, Urachne, Uralepis, Urochloa, Vahlodea, Valota, Vaseyochloa, Ventenata, Vernonia, Vetiveria, Vicia, Vigna, Vilfa, Vitis, Vulpia, Willkommia, Yushania, Zea, Zizania, Zizaniopsis, and Zoysia.

Additionally, the invention is also directed to a seed for a plant having a greater amount of aerenchyma. The plant can be made by identifying a plant having a greater amount of aerenchyma, as discussed above. The identified plant is used to produce the seed for the plant having a greater amount of aerenchyma by sexually or asexually reproducing the plant.

It was also unexpectedly discovered that greater amounts of aerenchyma also provide a plant with the ability to withstand drought. Thus, by determining the amount of aerenchyma in a plant and identifying whether the plant has a greater amount of aerenchyma, one can develop a plant that is more resistant to drought than other plants. If the plant has more aerenchyma, it will be more resistant to drought than plants that have less aerenchyma.

### EXAMPLES

The above described invention is further illustrated by the examples provided below. It is understood that the examples described herein are for illustrative purposes only and that various modifications or changes in light thereof will be suggested to persons skilled in the art and are to be included within the spirit and purview of this application and scope of the appended claims.

### Example 1

The inventor has surprisingly discovered that plants which develop more aerenchyma under flood conditions are also more tolerant to drought because they are better at capturing water. The inventor planted several inbred lines of maize (Zea mays L.) under flooded conditions to identify/confirm the lines that develop high amounts of aerenchyma and those that develop low amounts of aerenchyma in response to flood conditions. The difference in aerenchyma between the high and low aerenchyma lines would also be observable if the plants were grown under well-watered conditions. The same inbred lines were planted under drought stress. Surprisingly, the plants with high aerenchyma phenotypes were grew better under drought conditions, and thus, were more drought tolerant than the plants with low amounts of aerenchyma.

Materials and Methods

Six recombinant inbred lines (“RILs”) of maize (Zea mays L.), numbered 33, 34, 248, 284, 331 and 364 were obtained. These RILs were characterized as having either high or low root cortical aerenchyma (RCA) when placed under flooded conditions. RILs 33, 248 and 331 had low RCA, and RILs 34, 284 and 364 had high RCA under water stress.

The greenhouse experiment was a randomized complete block design with a two×six factorial arrangement of treatments. The factors were two water regimes (water-stressed and well-watered conditions) and six genotypes (RILs 33, 34, 248, 284, 331 and 364), and four replicates staggered one day between replicates with time of planting as the block effect.

Seeds were selected for uniform size, surface-sterilized in 0.05% NaOCl for 15 minutes and imbibed for 24 hours in aerated 1 mM CaSO₄. The seeds were placed in darkness at 28±1° C. in a germination chamber for two days. Before transplanting, the endosperm was removed from the seedlings. Seedlings of similar size were transplanted to mesocosms consisting of PVC cylinders 15.7 cm in diameter and 155 cm high. The cylinders were lined inside with plastic sleeves made of 4 mil (0.116 mm) transparent hi-density polyethylene film, which were used to facilitate root sampling. The growth medium consisted of a mixture (volume based) of 50% medium size (0.5-0.3 mm) commercial grade sand, 25% horticultural size #3 vermiculite, and 25% peat moss. Twenty-nine liters of the mixture were used in each cylinder. Two days before planting, the cylinders were watered with five liters of a nutrient solution adjusted to pH 6.0 and consisting of (in mM): NO₃ (7000), NH₄ (1000), P(1000), K (3000), Ca (2000), SO₄ (500), Mg (500), Cl (25), B (12.5), Mn (1), Zn (1), Cu (0.25), Mo (0.25), and EDTA-Fe (25). Each cylinder received two plants, and after two days, they were thinned to one plant. The plants were grown in a temperature-controlled greenhouse in University Park, Pa. with a photoperiod of 14/10 hours at 28/24° C. (light/darkness). Maximum midday photosynthetic flux densities reached 1200 mmol photonsm-2 s-1. The relative humidity was 40%. The well-watered treatment consisted of daily irrigation of 50 mL of deionized water for 5 weeks. In the water stress treatment, there was no irrigation for five weeks to progressively reduce soil water content. When plants were harvested five weeks after transplanting, soil water potential was 0.0 MPa at 10, 70, and 110 cm depths in well-watered mesocosms. In water stress treatments, soil water potential reached−0.91±0.04 MPa at 10 cm depth, −0.34±0.02 MPa at 70 cm depth, −0.10±0.02 MPa at 110 cm depth (n=24).

Measurements of root segment respiration were made in seminal roots. The sample was taken 15 cm from the base and consisted of a 15 cm long sample with the root laterals removed by a Teflon blade. Excised segments were patted dry and placed in a 40 mL custom chamber connected to the Li-6250 IRGA. The container with the sample was kept at 22° C. in a water bath for 2 minutes while the respiration was measured. Following respiration measurement, samples were stored in 25% ethanol for anatomical analysis as described as follows.

Root cross sections were obtained using a protocol that consisted of free-hand sectioning with Teflon-coated razor blades of root segments resting on dental wax inside a drop of water, with the aid of a dissecting microscope. The sections were observed with a Nikon Diaphot inverted microscope, the images were acquired using ImageMaster (Photon Technology International, Birmingham, N.J., USA) and analyzed in MATLAB 7.62008a (The MathWorks Company, Natick, Mass., USA), using code we developed for this purpose. The code consists of three phases. Phase I of the analysis involves separating the cross-section from any debris and from the background of the image. In Phase II, a series of steps isolates the stele from the cortex, allowing for separate analyses of these areas. In Phase III, data collection includes area measurement of desired features. The correct identification of these features is based on number of pixels and value thresholding for edge detection. The area of each cross-section and aerenchyma lacunae were obtained by pixel counting. The measurements were in mm², and were calibrated from pixels using an image of a 1 mm micrometer taken at the same magnification as the analyzed images. The percentage of aerenchyma as area of the cortex was estimated for four different sections per replicate.

The roots were separated from the soil by submerging cylinders in a container filled with water and rinsing the roots carefully in deionized water. Roots were separated from every 20 cm soil depth. The total root length in various soil depths were obtained by scanning with image analysis software (WinRhizo Pro, Régent Instruments, Québec, Canada). The maximum root length of each plant was recorded at 35 days after planting (“DAP”). The shoots and roots were dried at 60° C. for 72 hours prior to dry weight determination.

Results

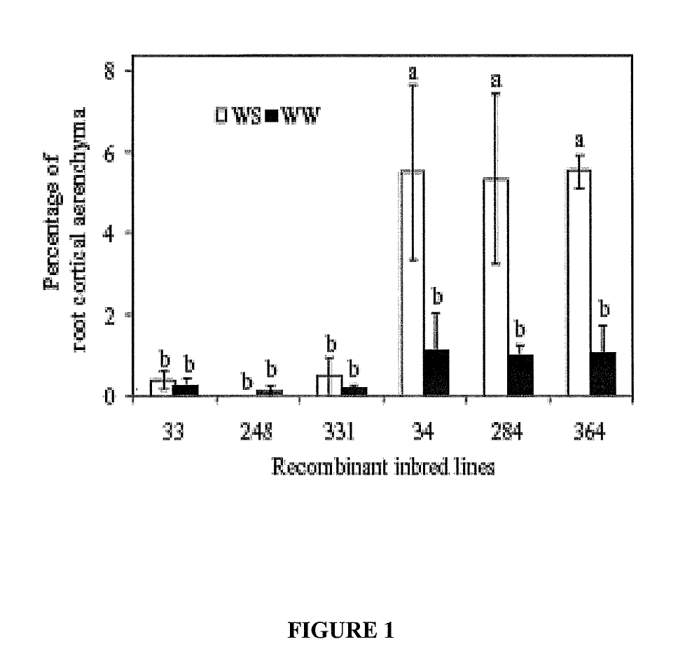

Water stress increased RCA of the six lines by an average of 382% at 35 DAP in mesocosms (FIG. 1). In water stress, lines 34, 284, and 364 (high RCA lines) averaged 5.5% RCA, while lines 33, 248, and 331 (low RCA lines) averaged 0.29% RCA. Under these stressed growing conditions, a plant would be considered to have high aerenchyma if it has between 2-80% aerenchyma.

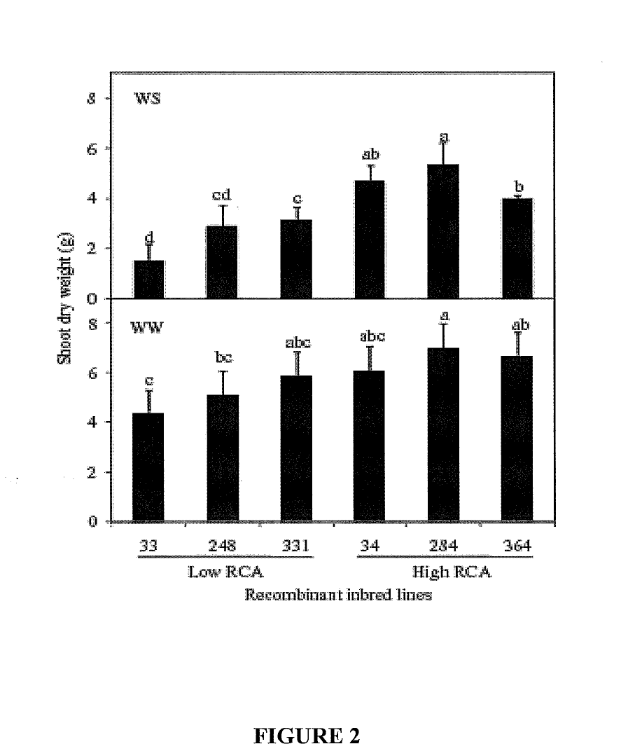

Water stress reduced the shoot biomass for each of the six lines by 39% at 35 DAP in mesocosms (FIG. 2). Under water stress, the three lines with high RCA had 86% more shoot biomass at 35 DAP compared to the three lines with low RCA (P≦0.05) (FIG. 2).

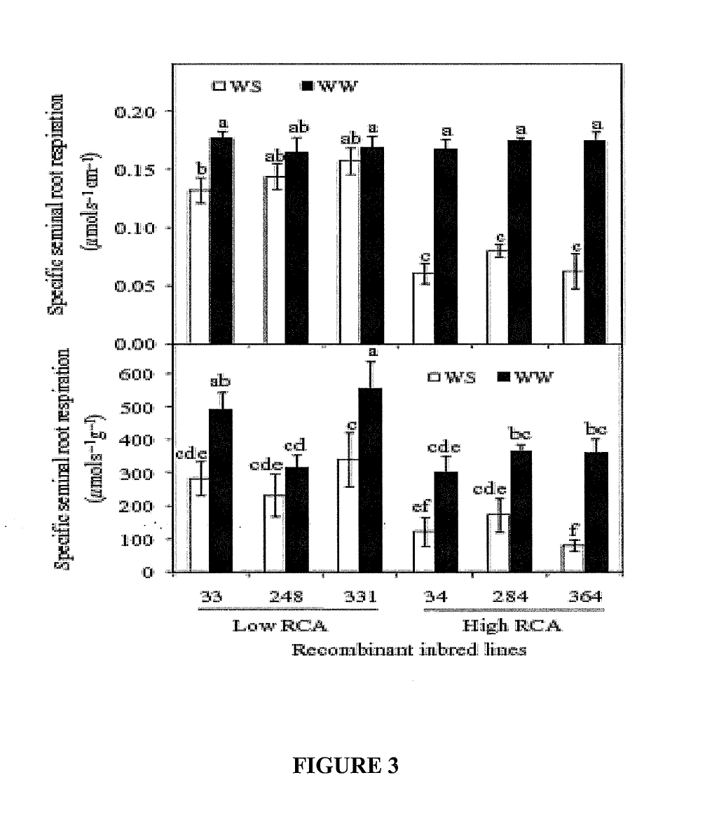

Water stress reduced specific root respiration per unit of root length and weight by 61% and 94%, respectively (FIG. 3). In water stress, the high RCA lines had 53 to 57% less specific root respiration per unit of root length and weight than the low RCA lines (FIG. 3).

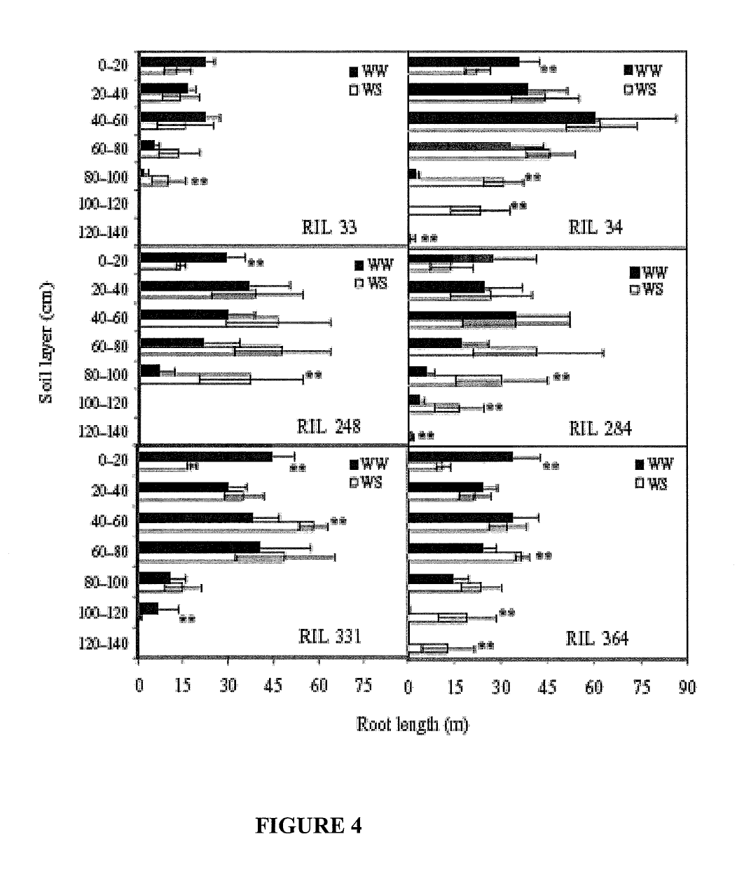

Water stress significantly increased root length density in deeper soil layers at 35 DAP in mesocosms (P≦0.05) (FIG. 4). Under water stress, the high RCA lines had significantly greater root length in 100-140 cm soil layers than the low RCA lines (FIG. 4).

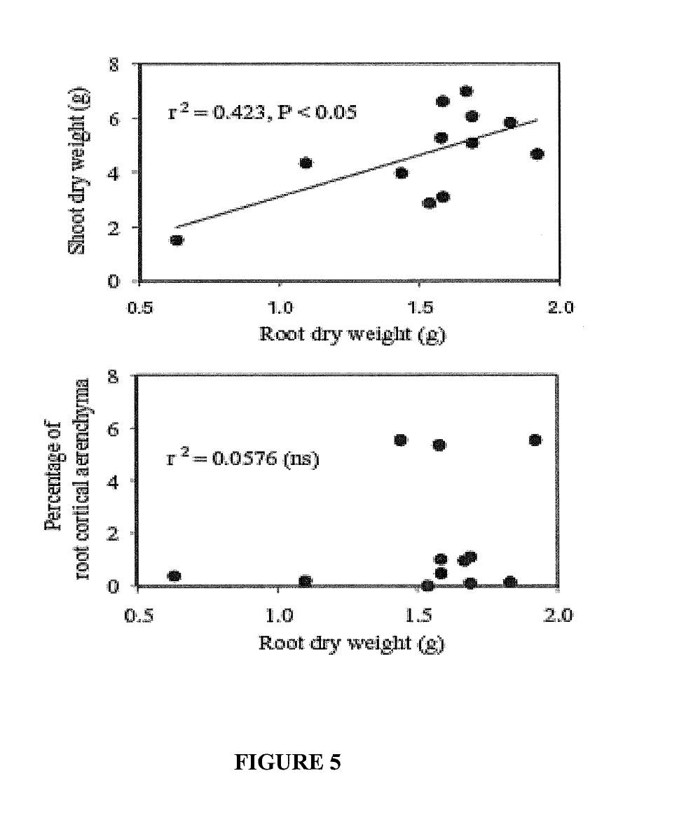

Root dry weight was significantly associated with shoot dry weight (FIG. 5), but root dry weight was not significantly correlated with percentage of root cortical aerenchyma for the six maize lines at 35 DAP in both well-watered and water stress conditions in the mesocosms. Covariate analysis showed root dry weight was non-significant for percentage of RCA (F=0.65, P=0.57) (FIG. 5).

Materials and Methods

The maize seeds of the six IBM RILs having contrasting RCA under water stress in mesocosms, plus RIL 77 having high RCA in a previous screening were planted under movable rainout shelters and in immediately adjacent non-sheltered plots at the Russell E. Larson Experimental Farm of Pennsylvania State University at Rock Springs, Pa. The experiment was a randomized complete block design with a split-plot arrangement of treatments. There were four biological replicates for each genotype, and each replicate had 30 plants grown in three 2.1 mrows. The shelters (10 by 30 m) were covered with clear greenhouse plastic film (0.184 mm) and were automatically triggered by rainfall to cover the plots and exclude natural precipitation throughout the entire growing season. The shelters automatically opened quickly after rainfalls, exposing experimental plots to ambient radiation and temperature conditions. Adjacent non-sheltered control plots were drip-irrigated as needed throughout the growing season to provide unstressed comparisons. The soil was a Murrill silt loam (fine-loamy, mixed, semi-active, mesic Typic Hapludult). The soil pH was 6.2, and the soil nutrient levels of N, P, K, Ca, Mg, Zn, Cu and S were optimum for maize production as determined by soil tests.

The soil volumetric water content was monitored with a multiplexed TDR-100 system (Campbell Scientific Inc., Logan, Utah, USA) spread randomly across plots and placed at 25 and 50 cm depths in the soil logged by a CR23X data logger (Campbell Scientific Inc.) in water stress and well-watered plots with four replicates throughout the growing season. The leaf relative water content (RWC) of the second fully expanded leaf was determined mid-day at 42 DAP according to the method of Barr and Weatherley (AU. J. OF BIOL. SC. (1962) 151: 413-428), which is incorporated herein by reference.

Root development with depth was monitored with minirhizotrons (BTC100X, Bartz Technology Co., Santa Barbara, Calif., USA). One clear acrylic minirhizotron root observation tube with a diameter of 5.1 cm was installed in each plot at a 30° angle from perpendicular to a depth of about 65 cm. The root length density and root number per frame were imaged from 10 to 50 cm depth in the soil with an interval of 10 cm at 42 DAP and analyzed by Rootfly software version 2.0. Destructive harvests at 56 DAP were used to measure root length and number density with depth by soil coring from 10 to 50 cm depth in the soil with an interval of 10 cm. The diameter of soil cores was 5.1 cm.

The roots and shoots were harvested at flowering. The RCA was sampled from the oldest nodal root in the maturation zone at 56 DAP with four subsamples per replicate. The tissue sectioning and RCA quantification were as previously described. The shoot dry weight was collected with three subsamples per replicate (n=4). At maturity, the individual seed number per plant and the individual seed dry weight were recorded to calculate an individual yield with three subsamples per replicate.

The data was analyzed using the Minitab statistical package (Minitab Inc., University Park, Pa., USA). The root depth, RCA, shoot and root dry weight, specific root respiration, relative water content, yield, individual seed dry weight, seed number per plant, root length and number per frame were first subjected to anova for main effects and first-order interactions using a general linear model that included water regime and genotype factors. The genotype and water regime were considered fixed effects, and replicates were random. Fisher's Least Significance Difference was used for multiple comparisons at P<0.05 level under post hoc anova.

Results

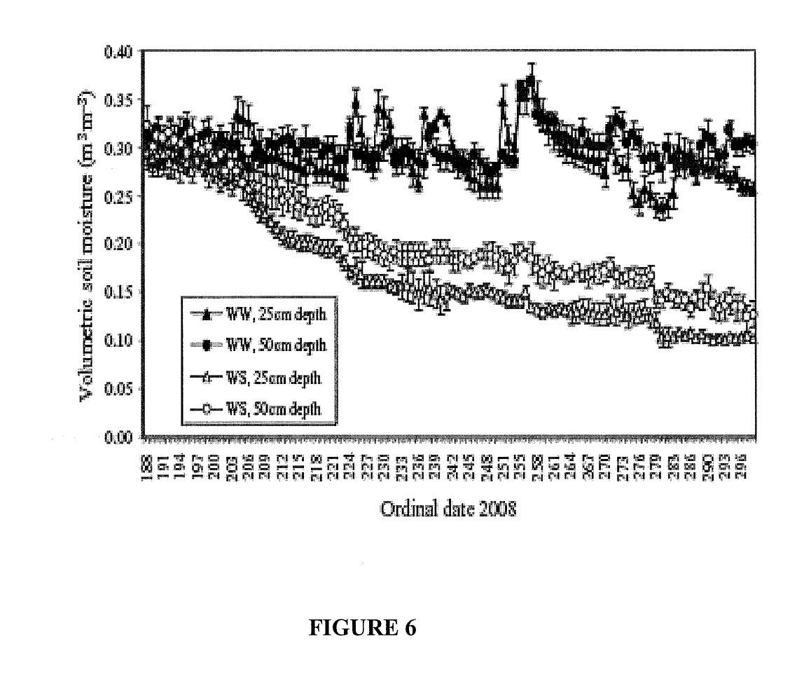

Volumetric soil moisture was maintained around 30% at both 25 cm and 50 cm depths in well-watered conditions throughout the growing season (FIG. 6). For water-stressed plants, volumetric soil moisture progressively decreased from 30% to 10% at 25 cm depth, and to 15% at 50 cm depth in rainout shelters (FIG. 6).

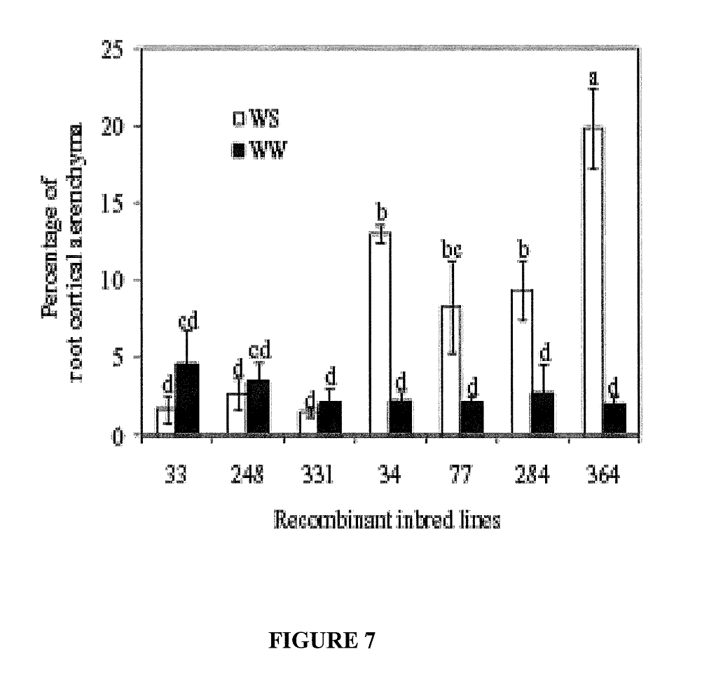

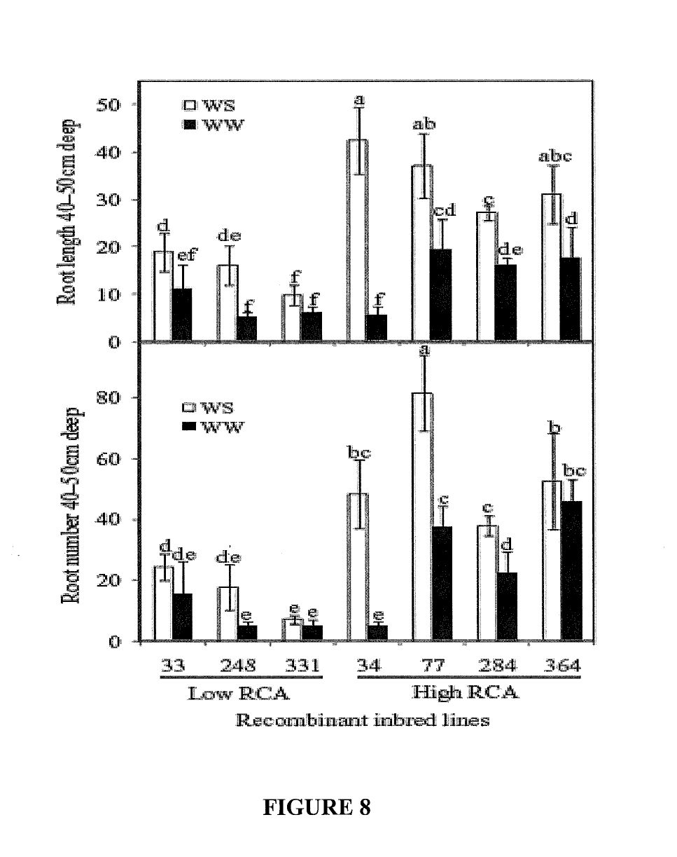

The water stress increased RCA in the high RCA lines at 56 DAP in the field (FIG. 7). Lines 34, 77, 284 and 364 (high RCA lines) averaged 12.6% RCA, while lines 33, 248 and 331 (low RCA lines) averaged 1.98% RCA. The water-stressed plants had doubled root length density and root number density at 40-50 cm depth in the soil compared to well-watered plants at 56 DAP in all seven lines (FIG. 8). Under these stressed growing conditions, 5-80% aerenchyma would be considered a plant having a high amount of aerenchyma.

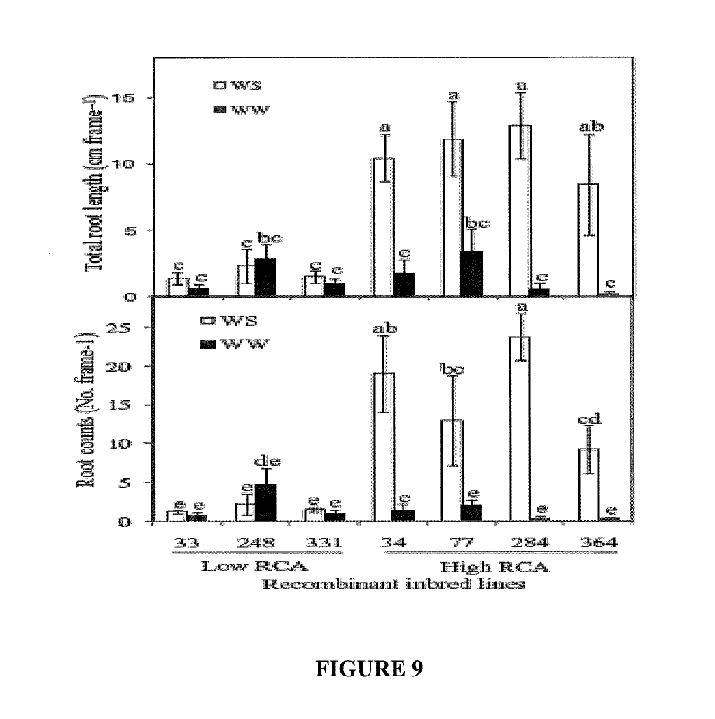

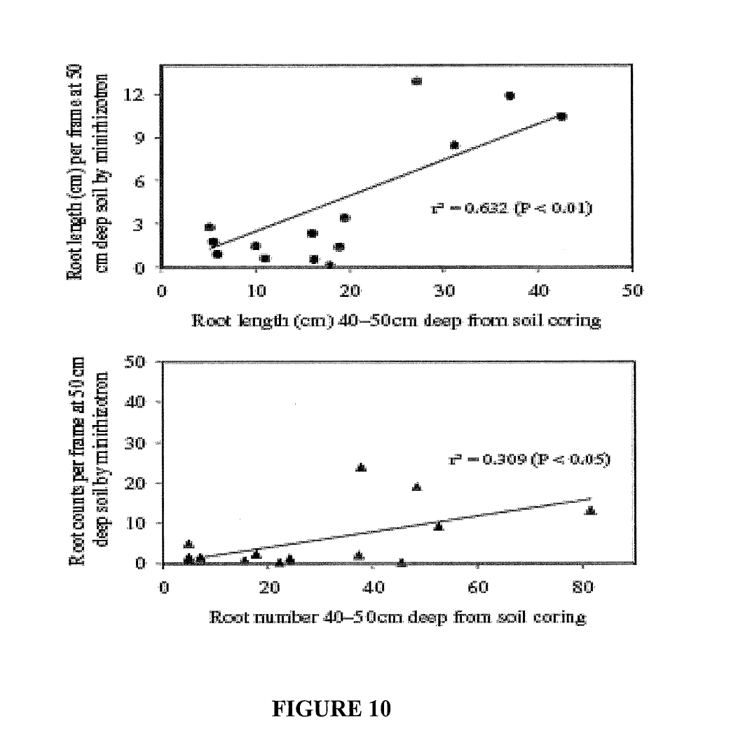

The root length density at 40-50 cm depth was significantly greater in the four high RCA lines compared to the three low RCA lines at 56 DAP (FIG. 8). The four high RCA lines averaged 3.4 times the root number density at 40-50 cm depth compared with the three low RCA lines under water stress (FIG. 8). The results from minirhizotron measurements further confirmed that high RCA lines had deeper rooting in terms of root length per frame and root number per frame compared to the low RCA lines at 42 DAP (FIG. 9). There was significant correlation between root length density measured by soil coring and by the minirhizotron technique (R2=0.63, P<0.01) (FIG. 10). Root number density was also significantly correlated (R2=0.31, P<0.01) between soil coring and minirhizotron technique (FIG. 10).

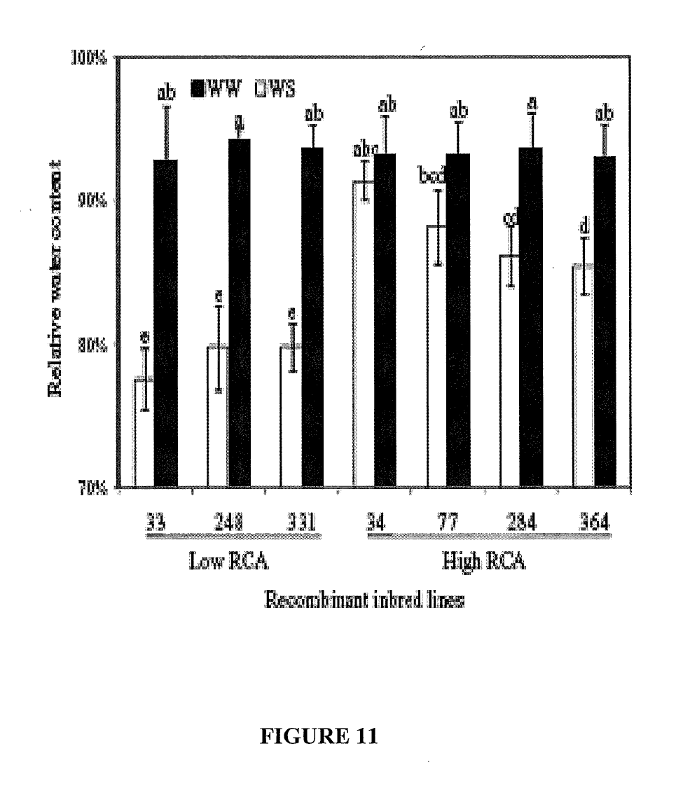

Mid-day leaf relative water content (RWC) at 42 DAP in the high RCA lines was significantly greater than for the low RCA lines in water-stressed conditions (FIG. 11).

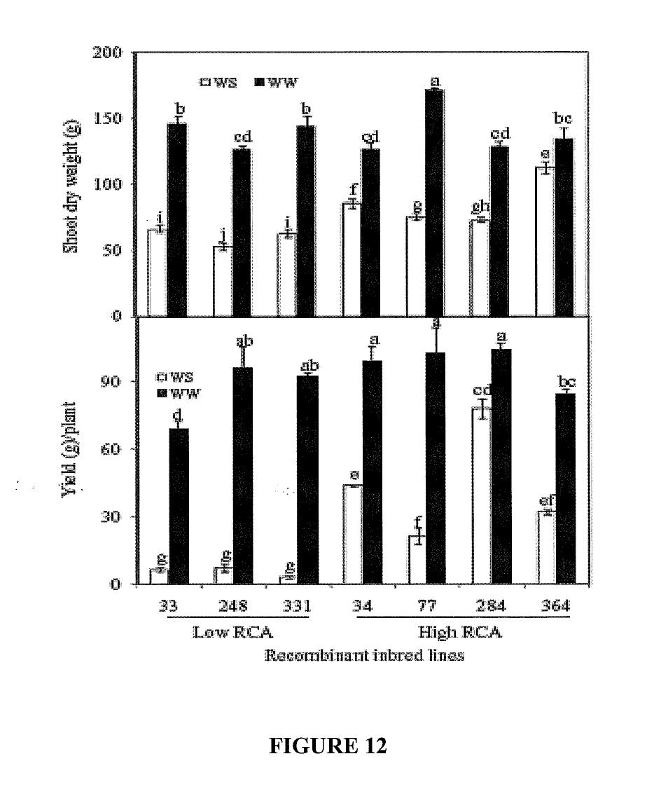

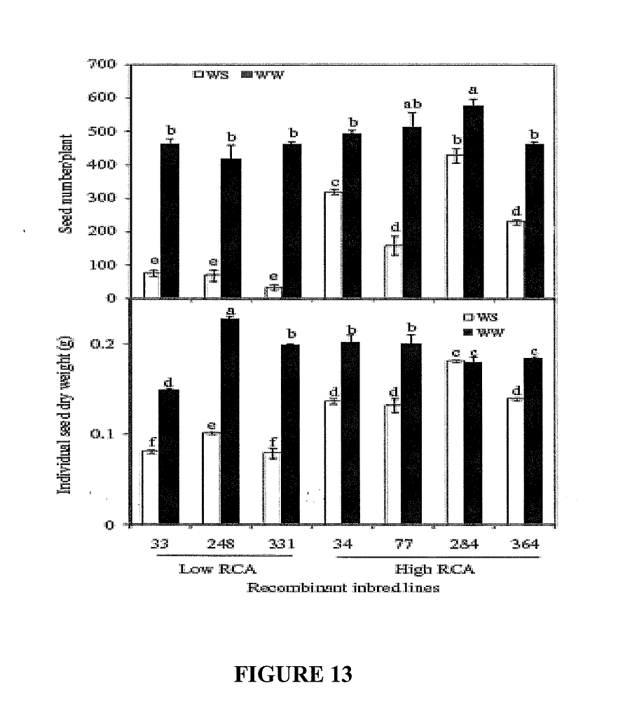

Shoot biomass of the seven lines was reduced by an average of 46% under water stress at flowering stage compared to well-watered plants in the field (FIG. 12). Under water stress, the four lines with high RCA had 43% more shoot biomass at flowering compared to the three lines with low RCA. The high RCA lines averaged eight times the yield of the low RCA lines under water stress (P<0.001) (FIG. 12). The increased yield under water stress was caused by increases of both kernel numbers per plant and individual kernel weight (FIG. 13).

Discussion

The results show that RCA increases drought tolerance by reducing root metabolic costs, permitting greater root growth and water acquisition. In mesocosms under controlled water stress conditions, three high RCA lines had lower specific root respiration, greater maximum root depth and greater shoot biomass than three low RCA lines. In the field under water stress conditions in rainout shelters, four high RCA lines had greater root depth, greater leaf relative water content and substantially greater grain yield than three low RCA lines.

RILs were used for the analysis of the physiological function of RCA, which permitted the comparison of closely related genotypes with identical genetic backgrounds without artificially induced mutations or transformation events. Each RIL was a distinct genotype, and comparison of six to seven RILs allowed the analysis of a phenotype in distinct genomes, thereby reducing the risk of confounding effects from pleiotropy, epistasis, or other genetic interactions. RILs were particularly valuable in the analysis of phenotypic traits governed by multiple genes, as RCA in maize.

Mesocosms in a greenhouse and movable rainout shelters in the field were used to obtain progressive reduction of soil water content, as typically occurs in seasonal drought. Plant biomass and yield were significantly reduced by the imposed stress treatment (FIGS. 2, 12 and 13). Significantly deeper rooting was observed in terms of root length density and root number density in water-stressed compared to well-watered plants of all genotypes in the two different culture systems (FIGS. 4, 8 and 9), as evidenced by soil cores and minirhizotron measurements (FIGS. 8 and 9). Deeper rooting in response to water stress enhances water acquisition, since deeper soil domains generally have greater water content compared to shallow soil domains, as evidenced in this study by time domain reflectometry (TDR) measurements in the field (FIG. 6) and soil water potential measurements at 35 DAP in mesocosms.

Root metabolic costs are an important component of plant growth and adaptation under low phosphorus availability. Root cost can be estimated as total below-ground expenditure of carbon, which is used for root construction, root maintenance, and ion uptake. Specific root respiration per unit of length and weight were significantly reduced by water stress (FIG. 3). Higher RCA lines have more cortical air space in root tissues (FIGS. 1 and 7), which may reduce root carbon cost for root maintenance in higher RCA lines. Thus, higher RCA lines may have more carbon and energy available for root construction, permitting exploration of deeper soils under water stress (FIGS. 3, 4, 6, 9 and 10). Covariate analysis showed that there was no significant relationship between total root dry weight and percentage of RCA (F=0.65). In other words, RCA was not driven by the size of root system or the size of the plant (FIG. 5). High RCA lines with deeper rooting at water stress had higher leaf relative water content (FIG. 11). These findings demonstrate that RCA plays an important role for plant tolerance to drought.

The seminal root respiration per unit length (y₁) was significantly associated with percentage of RCA under water stress (x) (FIGS. 1 and 3). The linear regression formula was:

y₁=−0.014x+0.148 (r²=0.94, P<0.001).

A similar correlation was found for seminal root respiration per unit weight (y₂) and percentage of RCA under water stress (x) (FIGS. 1 and 3). The linear regression formula was:

y₂=−30.4x+293 (r²=0.77, P≦0.05).

These indicate about 6% RCA in seminal root halves, root respiration per unit length or weight under water stress. Twenty percent RCA cuts root respiration per unit volume in half at low phosphorus availability in a solution culture study.

In maize, an additional benefit to reducing root costs is that yield losses to drought are related to carbohydrate availability during reproductive growth. Reduced root carbon demand in high RCA genotypes may be beneficial by increasing carbohydrate availability during reproductive growth, since reproduction and roots are competing sinks for current photosynthate (FIGS. 3, 12 and 13).

The water stress induced greater expression of RCA in drought-tolerant lines in both seminal roots of five-week-old plants in mesocosms and nodal roots of eight-week-old plants in the field, compared to the sensitive lines (FIGS. 1, 2, 7, 12 and 13), although the absolute values of increased RCA differed in various root types. The seminal roots of five-week-old plants and nodal roots of eight-week-old plants are major components of root system architecture. This response to the water stress allows more carbon allocation for root exploration into deeper soil, greater water acquisition, and greater shoot biomass and yield under stress (FIGS. 2, 3, 4, 6, 8, 11, 12 and 13). Collectively, these indicate (1) an increase of RCA in different root types at various developmental stages in water stress to be a positive adaptation for plants; and (2) the magnitude of RCA responses to water stress may be related to root type, plant developmental stage, and drought severity.

There was substantial genetic variation for RCA among seven maize genotypes under water-stressed conditions. The results demonstrate that RCA reduces the metabolic costs of soil exploration, leading to greater water acquisition in drying soil. Thus, plants having a greater amount of RCA will have improved drought tolerance. To this end, breeding programs to develop plants, such as maize, having higher amounts of RCA can be used to develop lines that are drought tolerant.

### Example 2

SimRoot, a functional-structural plant model, was used to evaluate the potential importance of root cortical aerenchyma (RCA) as an adaptation to low phosphorus availability in a quantitative manner. SimRoot is a mechanistic model which allowed us to evaluate the quantitative relevance of the two proposed functions of RCA formation in phosphorus deficient maize and bean. The results discussed below can be used to support breeding programs for soil mineral (for example, primary macronutrients) efficient plants, such as maize and bean. This assessment of the potential benefits of RCA formation includes the effect of elevated CO₂, since it is expected that atmospheric CO₂ concentrations will continue to rise. A high CO₂ environment may increase photosynthetic rates, especially in C3 plants, as photorespiration is reduced under elevated CO₂. Greater carbon assimilation may decrease the relative benefit of carbon saving strategies like reduced respiration, while phosphorus remobilization from the cortex may become more significant.

The formation of RCA reduces root respiration and nutrient content by converting living tissue to air volume. The inventor has discovered that RCA increases soil resource acquisition by reducing the metabolic and phosphorus cost of soil exploration. This was demonstrated, in part, through the use of SimRoot, a functional-structural plant model with emphasis on root architecture and nutrient acquisition. Sensitivity 7 analyses were conducted for the effects of RCA on the initial 40 days of growth of maize (Zea mays L.) and common bean (Phaseolus vulgaris L.) in soils with varying phosphorus availability. With reference to future climates, the benefit of RCA in high CO₂ environments was simulated.

The model shows that RCA may increase the growth of plants faced with suboptimal phosphorus availability up to 70% (maize) and 12% (bean) after 40 days of growth. The maximum increases were obtained at low phosphorus availability, 3 μM. Of the two proposed functions, remobilization of phosphorus had the largest effect on plant growth. Both functions were additive. The benefit of RCA increased over time and larger benefits may be expected for mature plants. Sensitivity analysis for light use efficiency showed that the benefit of RCA is relatively stable. This suggested that elevated CO₂ in future climates will not significantly affect the benefit of RCA.

These results demonstrate an adaptive trait for phosphorus acquisition by remobilizing phosphorus from the root cortex and reducing the metabolic costs of soil exploration. The benefit of RCA in low phosphorus soils is larger for maize than bean, as maize is more sensitive to low phosphorus availability while it has a more expensive root system. The genetic variation in RCA may be useful for breeding phosphorus-efficient crop cultivars, which is important for improving world food security. Based on these results, it is expected that higher amounts of RCA would also increase the acquisition of other soil resources, including nitrogen and potassium.

SimRoot, originally a root architectural model, was augmented into a whole plant model with emphasis on resource acquisition and utilization. Two resources considered were phosphorus and carbon. An RCA module for SimRoot parameterized for both common bean (Phaseolus vulgaris L.) and maize (Zea mays L.) was developed. Simulation of root architecture by SimRoot has been described by Lynch et al. (1997). In SimRoot, the root system is represented by connected root nodes, spaced approximately 0.5-1 cm apart. Important root properties, for example, phosphorus uptake, are calculated for each root node and integrated over the length of the root system. Shoots were not geometrically simulated, but rather the shoot was represented as a canopy with a number of resource pools, sources and sinks.

Carbon Module

The carbon module defines carbon sources and sinks and a set of allocation rules. Seed reserves and photosynthetic CO₂ assimilation are the two carbon sources. Carbon availability from seed reserves is based on 1) the initial seed dry weight; and 2) an on-demand release function. A constant conversion factor between dry weight and carbon is used in the whole model. Photosynthesis is simulated as in the LINTUL (Light Interception and Utilization simulator) model. Growth of leaf area is based on carbon allocation to leaves and the specific leaf area. Leaf area is converted to leaf area index (LAI) using a user specified area per plant. The intercepted light is calculated using the following formula in which K is a crop specific extinction coefficient and PAR is the photosynthetic radiation: intercepted light=par*(1.-e^{(k*LAI)}).

Intercepted Light is Converted to Photosynthesis Using a Light Use Efficiency Factor.

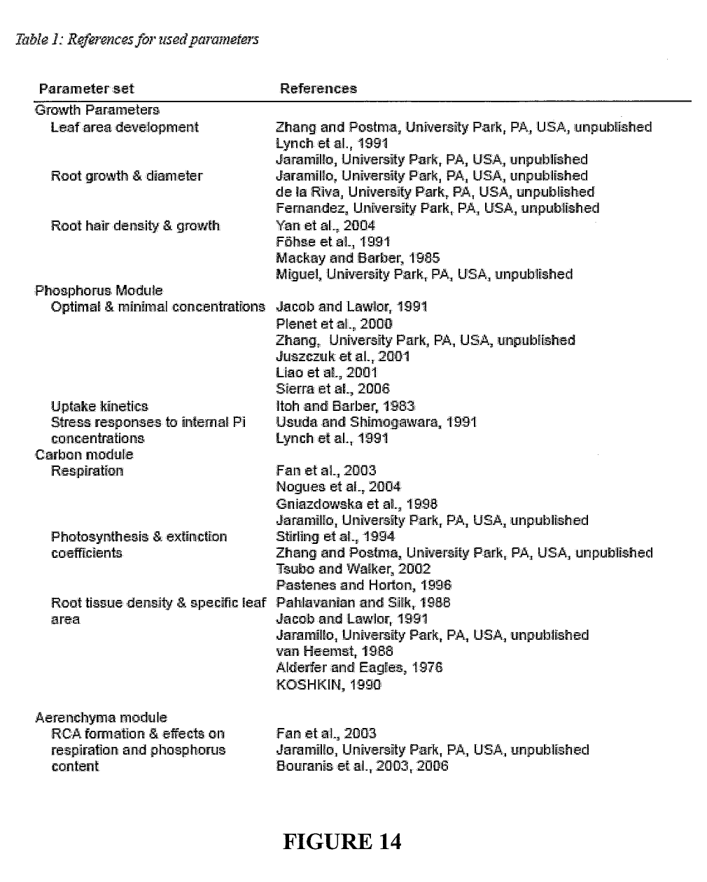

Sink strength in SimRoot is based on the carbon needed for potential growth, respiration, and exudates. Potential growth is based on measured growth rates for all root classes, and measured relative growth rates of leaves and stems (FIG. 14). Thicker roots have higher longitudinal potential growth rates and therefore, larger sink strength. Carbon needed for secondary growth is calculated by the volumetric increase needed for potential secondary growth. Potential secondary growth depends on the age, distance along the root and the root class of each root segment. Respiration is calculated as a function of organ biomass and age. Separate respiration coefficients were used for each organ (FIG. 14). There was no explicit distinction made between growth respiration, respiration caused by exudates and maintenance respiration. Maintenance respiration forms the largest portion of respiratory costs, while respiration associated with exudates forms less than 3.5% of the daily carbon budgets of common bean. Maize produces even less exudates. Growth respiration is implied in the respiration function, as the root apical meristems have a much higher respiration coefficient in the model (FIG. 14). The higher respiration of meristems is not affected by RCA, as RCA only forms in older root segments. The third sink, carbon cost of exudates, is calculated based on root class and root age using empirical data (FIG. 14).

The carbon partitioning rules were based on a hierarchical binary partitioning method where sink strength, priority, and limits determine the partitioning between two sinks. First, carbon is partitioned between growth and non-growth sinks. The non-growth sinks are exudates and respiration, which are both considered obligate costs. Secondly, carbon is partitioned between shoot and root, with the shoot receiving priority over the root system. However, limits are set on the relative allocation to the shoot. The carbon allocated to the shoot is partitioned between stems and leaves proportional to sink strength. The carbon allocated to the root is partitioned between primary and secondary growth. Secondary growth is given as much carbon as is needed to maintain the allometry between secondary growth and leaf area. Thus, the model simulated root etiolation, which is a reduction in secondary growth of beans grown in soils with low phosphorus availability. The remaining carbon for root growth is allocated to primary growth of the root system. Carbon for primary growth is divided over the major axes and the fine roots. The major axes, within limits, are given priority over the fine roots. For maize, the number of nodal and brace roots were scaled allometrically according to the leaf area at the emergence of the roots. Within each group of root classes, carbon is allocated to the root tips proportional to their relative sink strength. The model includes temporal carbon storage, which increases when the available carbon for growth exceeds the carbon that is required for potential growth. This storage will act as a carbon source when the potential carbon requirements cannot be met, and as long as the storage contains carbon.

The carbon module includes positive and negative feedbacks over time, as the root and shoot growth led to changes in sink strength and resource capture. Therefore, a predictor-corrected integration method, Runge-Kutta 4, was used for computational efficiency and numerical accuracy. A carbon balance check was included to verify the consistency of the model by comparing the cumulative carbon expenditure with the cumulative carbon assimilation. For the carbon balance, organ dry weights were recalculated from their geometric dimensions.

Nutrient Module

Nutrient uptake by each root node was simulated using the Barber-Cushman model (MODELING WASTE WATER RENOVATION (1981): 382-409), including root hairs as described by Itoh (PLANT AND SOIL (1983) 70: 403-413), which are incorporated herein by reference. The average mid-distance between the roots in the vicinity of each root segment was used as the outer boundary for the Barber-Cushman model, across which the nutrient flux 9 was assumed to be zero. The mid-distance between root segments is adjusted each time a new root grows into the vicinity of other roots. The initial nutrient concentration to which new roots are exposed is corrected for nutrient depletion by other roots. The uptake rate of all root nodes is integrated over the length of the root system and over time to calculate the total nutrient uptake by the plant. The plant is given an initial amount of nutrients from the seed reserves.

The optimal and minimal nutrient concentrations are used to compute target nutrient levels in the plant. A stress factor is then calculated based on the actual uptake in comparison to minimum and optimal nutrient homoeostasis. This stress factor is used to adjust potential leaf area expansion rate and photosynthetic efficiency of the leaves. Phosphorus deficient plants have smaller leaves and slower leaf appearance. The stress factor is used to reduce the potential leaf area expansion rate, thus reducing the sink strength of the shoot and increasing relative allocation of carbon to the root system. It was assumed that the photosynthetic efficiency of the leaf area is reduced up to 50% for both maize and bean. Therefore, a sensitivity analysis for this reduction in light use efficiency was included. In the model, the respiration rates were not affected by the plant's internal phosphorus status.

Seven RCA Module

RCA formation is computed for each root node, based on developmental age. This information is used to simulate a reduction in root respiration and to reduce the optimal and minimal nutrient content of the root.

Runs

All simulations were run for 40 days of growth after germination. Full factorial designs were run, which included two plant species: maize (Zea mays L.) and common bean (Phaseolus vulgaris L.), varying amounts of RCA formation, the two proposed RCA functions separately and in combination, and varying amounts of nutrient availability. A sensitivity analysis for phosphorus availability, percentage RCA formation, light use efficiency and effects of phosphorus status on light use efficiency was conducted.

Parameterization

Parameterization of the model was based on empirical data of several experiments and the literature (FIG. 14). No parameters were obtained by calibration. It was assumed that root classes were identical when root class specific measurements were lacking.

Results

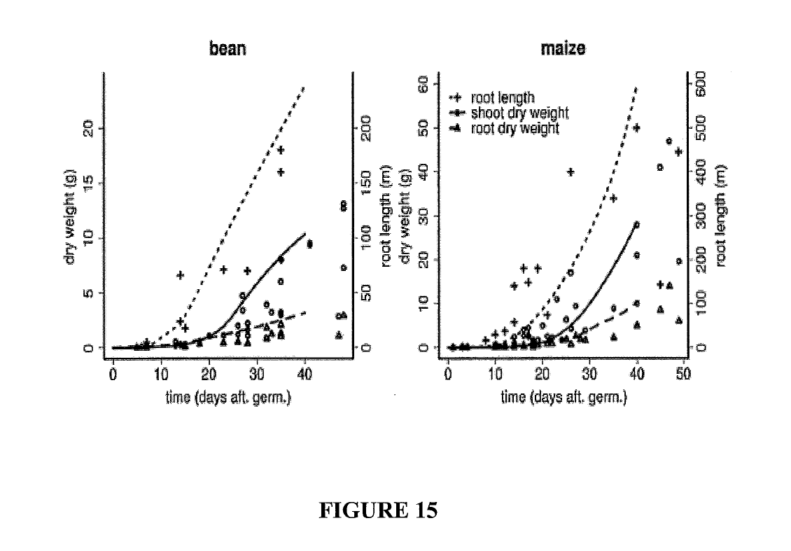

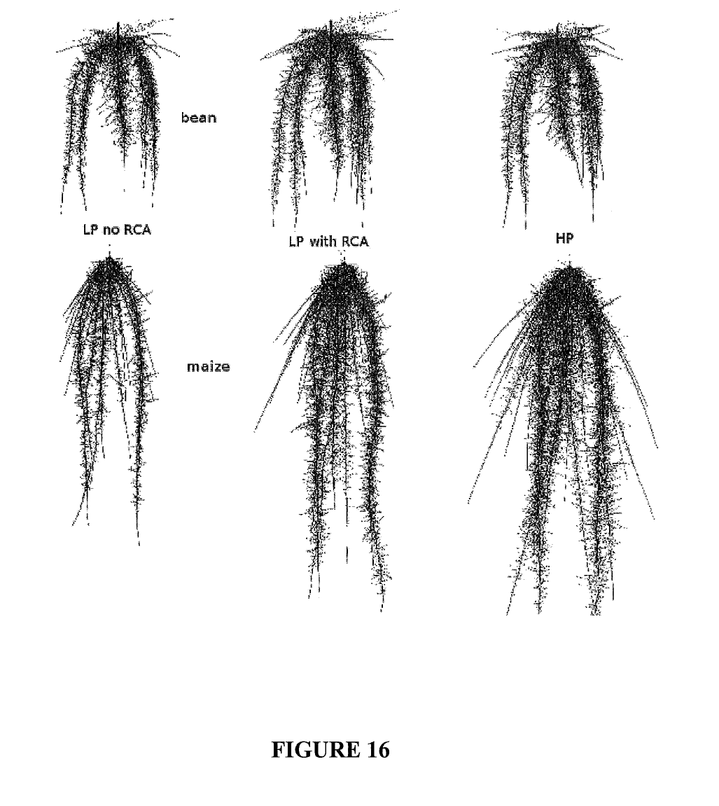

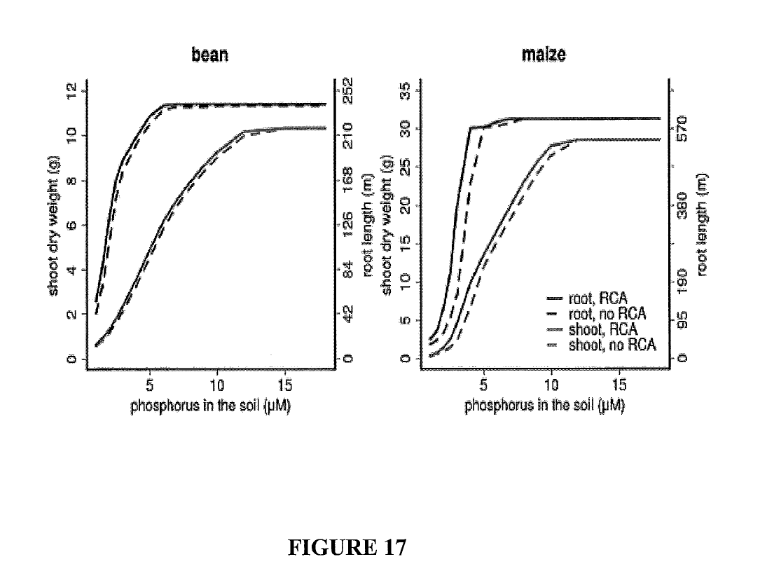

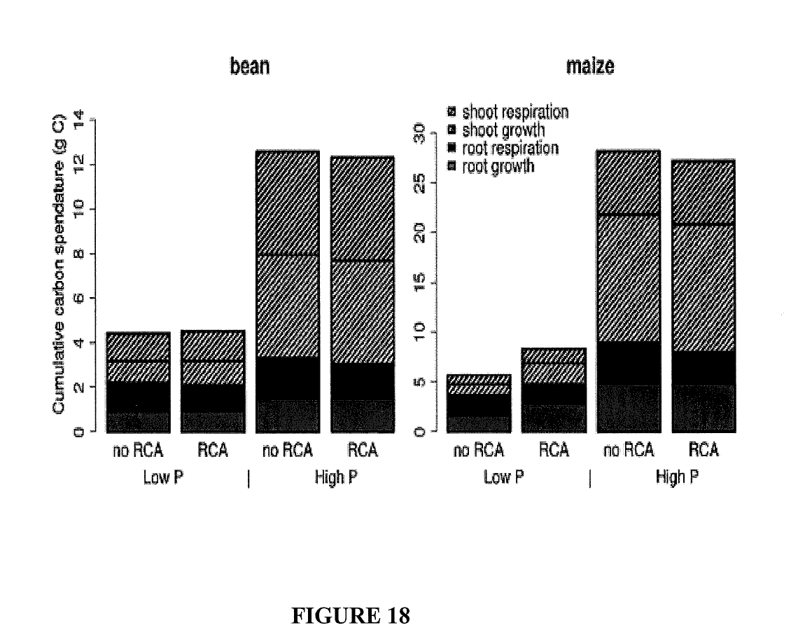

The model results were compared against independent data set (FIG. 15). The model results agreed well with the independent data set, although the root dry weight of both bean and maize is relatively high in the model results. The simulated bean plants were smaller than the simulated maize plants (FIG. 16), but had relative to their size, longer root length with more fine roots (FIG. 17). In maize, root development is centered on the growth of new major root axes, especially nodal and crown roots; while in bean, root development is centered on secondary growth and the extension of the initial axes and lateral roots. Both plants allocated more carbon to the shoot than the root. This, however, was more so in bean than in maize (FIG. 18). The slower growing bean plants spent relatively more energy on respiration. Respiration became an increasing cost over time as relative growth rates decreased and total plant biomass increased. Phosphorus deficiency increased the root/shoot ratio, significantly reducing plant growth rate. Consequently, phosphorus deficient plants had relatively greater respiratory costs (FIG. 18).

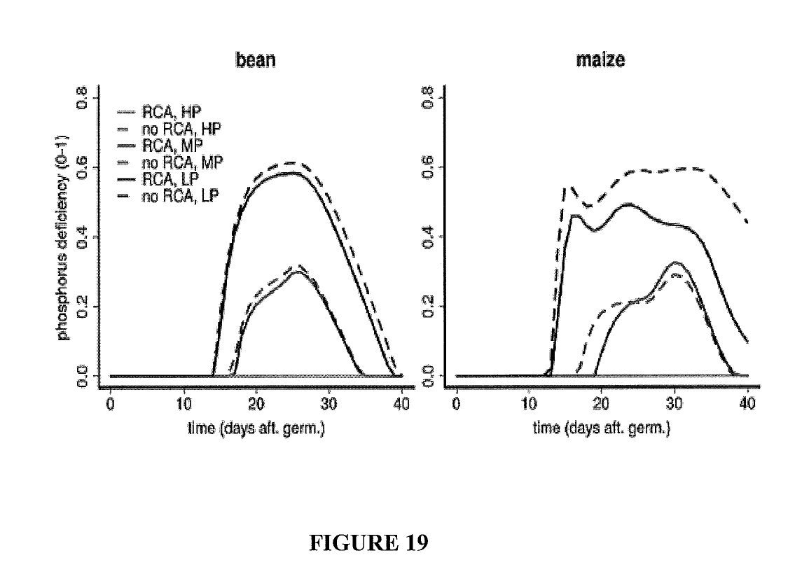

Plants responded to phosphorus deficiency with a sigmoid growth curve (FIG. 17). Relative biomass reduction in response to low phosphorus was less for bean than for maize, suggesting that bean is better at coping with phosphorus deficiency than maize. Plants were not deficient in high phosphorus soils, but in soils with reduced phosphorus availability deficiency occurred after 10 days (FIG. 19). At later stages, an improvement in the phosphorus status of the bean plants occurred, as altered root/shoot carbon allocation resulted in additional root growth and consequently phosphorus acquisition while carbon fixation was reduced. Maize plants only recovered from phosphorus deficiency in soils with medium phosphorus availability (6 μM).

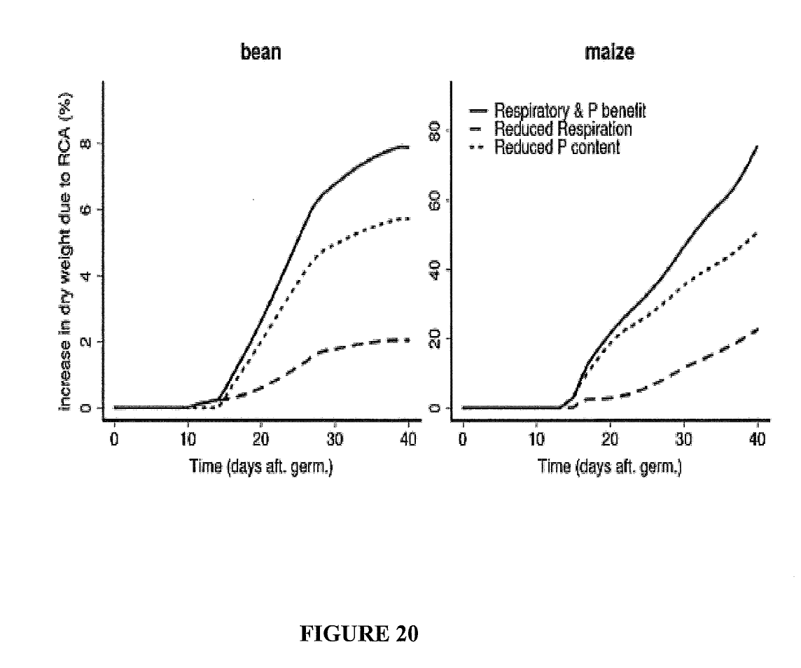

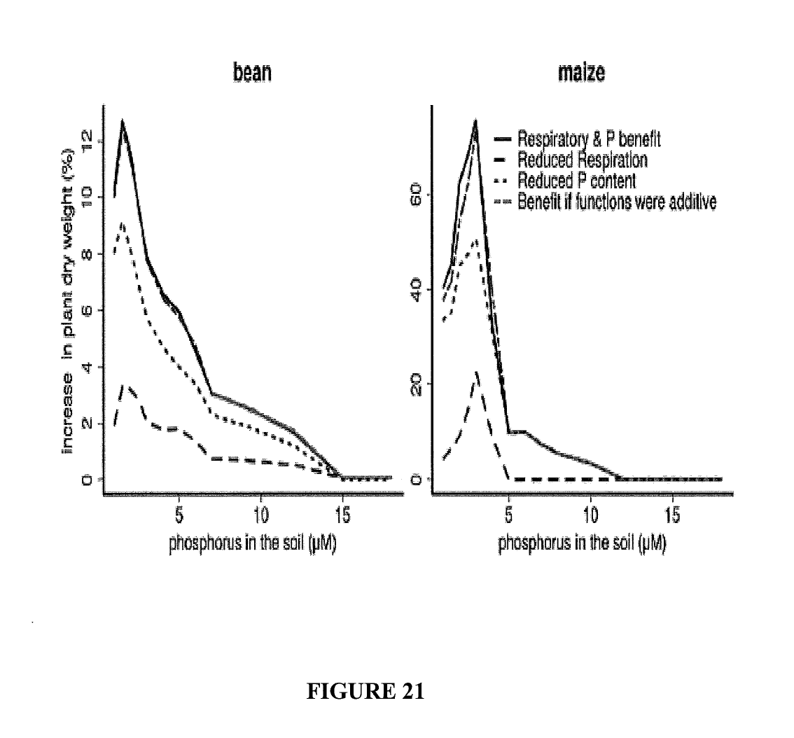

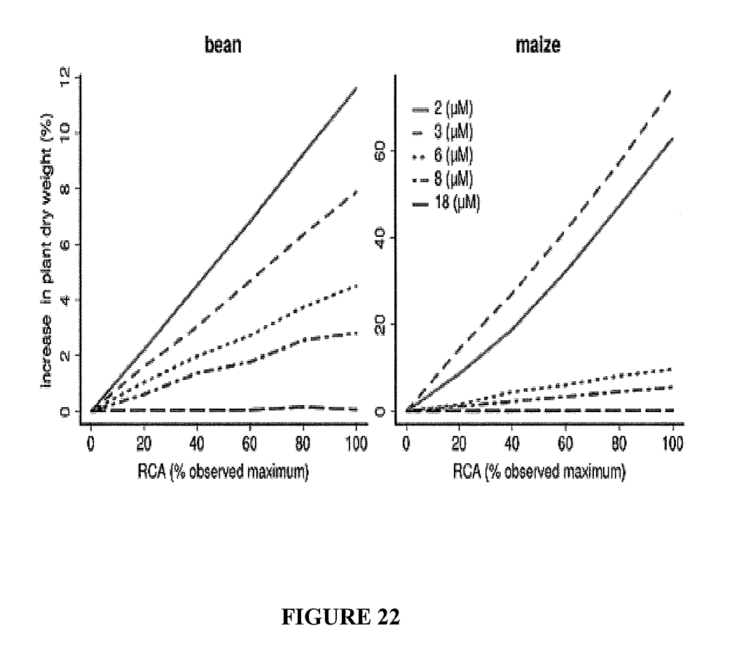

Plants with RCA were less stressed and grew faster under suboptimal phosphorus availability (FIGS. 17 and 19). The benefit of RCA increased with time (FIG. 20). After 40 days, phosphorus deficient plants with RCA were up to 12% (bean) and up to 70% (maize) larger than plants without RCA (FIG. 21). RCA had a small positive effect on the growth of non-deficient plants caused by a reduction in root respiration. The two proposed functions of RCA benefited plant growth to different degrees (FIG. 21). The most important function of RCA was remobilization of phosphorus from cortical tissue, which increased growth of phosphorus deficient maize plants up to 50%. Remobilization of phosphorus benefited shoot growth up to 60% and root growth up to 40%. Reduced respiration due to RCA formation had smaller effects on growth, increasing root growth up to 24% and, as a result of increased phosphorus uptake, shoot growth up to 20%. The root system of phosphorus deficient plants with RCA had lower relative respiration rates but not lower total respiration, as plants with RCA had larger root systems (FIG. 18). Presumably, the remobilization and respiration functions of RCA occur simultaneously, and the model suggests that the benefits of the two functions are additive. Sensitivity analysis showed a linear correlation of RCA formation and plant growth (FIG. 22).

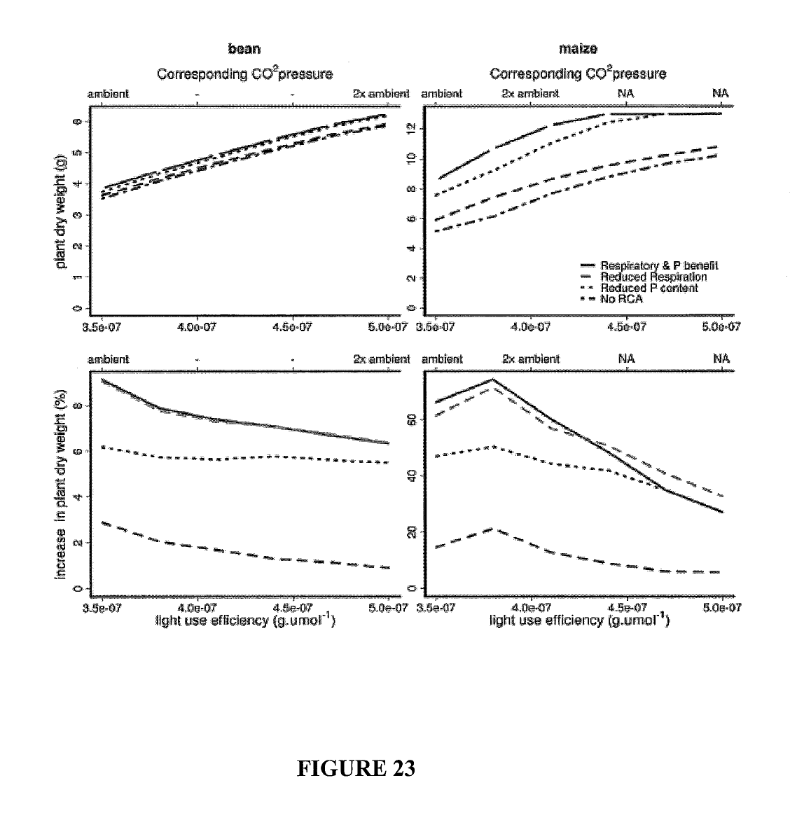

An increase in light use efficiency resulted in an increase in total biomass production of plants grown at 3 μM (FIG. 23), but affected plants grown at 18 as growth of high P plants was mostly sink limited. At low P availability (3 μM) the benefit of RCA decreased slightly with increasing light use efficiency (FIG. 23).

Discussion

SimRoot, a functional-structural model, mechanistically simulated the benefit of root cortical aerenchyma (RCA) in maize and bean plants under suboptimal phosphorus availability. In the simulations, RCA always had a positive effect on the growth of phosphorus deficient plants, increasing biomass production up to 12% for bean and up to 70% for maize (FIG. 21). The results demonstrate that RCA is an adaptive trait in soils with suboptimal phosphorus availability. Thus, RCA benefits plant growth through reduced respiration and remobilization of phosphorus. It is expected that RCA would provide similar benefits under other deficiencies in soil resources, for example, deficiencies in primary macronutrients.

RCA formation varies among and between species. Maize forms twice as much RCA as bean. However, maize plants with the same amount of RCA formation as bean plants still benefit nearly twice as much from RCA as bean plants do (FIG. 22, maize at 50%). Bean plants have a finer root system with more root hairs and are thereby less sensitive to phosphorus availability in the soil (FIG. 17). At a phosphorus concentration of 3 μM, the shoot growth of maize is reduced to 16% of potential, while for bean plants the shoot growth is at 30% of growth potential. The relative, but not the absolute, benefit of RCA for bean is thereby reduced. Furthermore, at these phosphorus availabilities, bean, with its finer root system and less reduced shoot size, spends relatively less carbon on root respiration, namely 15% of daily photosynthesis, versus 35% for maize. Therefore, a reduction in root respiration due to RCA formation in bean has less effect on the total carbon budget of the plant. Similarly, phosphorus remobilization from the roots has less effect on the phosphorus status of the plant because bean has a lower root/shoot ratio than maize (0.91 versus 1.5). Comparing maize and bean at similar shoot growth reductions is difficult, since the cost/benefit ratio for root growth strongly depends on phosphorus availability.

The results show that greater photosynthetic efficiency would increase plant growth at low phosphorus availability, and decrease the benefit of RCA formation (FIG. 23). The decrease in the benefit of RCA formation is a relative decrease, not an absolute decrease. The decrease is caused by the decreasing benefit of reduced respiration. Overall, the decrease in the benefit of RCA is small. Therefore, even under high CO₂ environments, RCA will still be beneficial.

The results show that RCA always increased plant growth, even when a plant is grown in non-deficient soil.

RCA formation is an adaptive trait in soils with suboptimal phosphorus availability. The functions of RCA under phosphorus deficiency are 1) reduced root respiration, allowing carbon to be used for greater soil exploration; and 2) remobilization of phosphorus, which is more important than reduced respiration in stimulating growth.

Low phosphorus availability is a major constraint to agricultural production, often reducing yields to less than 10% of yield potential. It is at these severe deficiencies where RCA has the most benefit. Significant genotypic variation in RCA formation exists in maize and bean. Such variation may be useful for breeding phosphorus efficient crops. Since RCA forms in many species in response to several nutrient deficiencies, RCA may be a positive trait for nutrient acquisition in general and could be part of a breeding strategy for more efficient crops as well.

### Example 3

Breeding for high levels of RCA formation results in crops with better growth in soils characterized by a several edaphic stresses, including drought, phosphorus deficiency, potassium and nitrogen deficiency.

RCA formation is a strongly plastic root trait. The amount of RCA that forms in the cortex depends on many factors including genetic, exogenous (environmental), and endogenous cues. As a result the amount of RCA that is formed in the cortex may differ between and within root classes of the same plant, and may vary along the length of a root segment. Quantitative information on local RCA formation is sparse and difficult to relate to exogenous or endogenous cues.

RCA leads to increased resource availability in plants. The benefit of RCA depends strongly on the existence of a positive feedback of increased resource availability on root growth and thereby nutrient uptake. Thus, the benefit of RCA is large when root growth is source-limited, not sink-limited. Stressed plants are often source limited as shoot growth is reduced and the root system becomes a relatively large burden. In moderately stressed plants (growth reduction up to 50%), however, root growth can be sink limited. Under these conditions, increasing the sink strength of the root system, by for example increasing the lateral rooting density, may improve the benefit of RCA.

Nitrate is a mobile resource that in agroecosystems often leaches into the subsoil during the growing season. The dynamics of nitrate leaching present challenges to root systems. Phosphorus and Potassium are often more available in the top soil and thus, in contrast to nitrate, top soil foraging may be more efficient.

Material and Methods

SimRoot was used to simulate the benefit of RCA formation in maize given different environmental conditions. RCA formation in three different maize genotypes was simulated wherein the amount of phosphorus, potassium or nitrate availability in the soil varied. For the nitrate study, a loamy-sand and a silt-loam soil and varied precipitation to create six different leaching environments was simulated. Synergism between RCA formation in lateral branching density was determined.

RCA formation in maize is simulated for each root segment using empirical data that is related to the root class and age of the root segment. RCA formation is allowed to either reduce the nutrient content of the root segments (reallocation function) or the respiration of the root segments (respiration function) or both, as is the case in real plants.

The nutrient stress factor was allowed to impact the potential leaf area expansion rate and the light use efficiency independently. A negative impact on light use efficiency reduced the carbon availability for growth. A negative impact on the potential leaf area expansion rate reduced the sink strength of the shoot, making more carbon available for root growth. It functions as a growth regulator altering root/shoot ratios. A nutrient specific stress response curve was used to determine the impact of the individual internal nutrient concentrations (Ni, Ki and Pi) on the light use efficiency and potential leaf area expansion rate. The impact of suboptimal Ki strongly affected light use efficiency, but was not a growth regulator. In contrast, suboptimal Pi strongly regulated growth, but had minor effects on the light use efficiency of the leaves. Nitrogen strongly affected both the potential leaf area expansion rate and the light use efficiency.

To simulate nitrate uptake, SWMS-3D was linked to SimRoot. SWMS-3D is a three dimensional hydraulic simulation model that includes a solute transport model. It simulates water transport in the soil by solving the Richard's equation, and solute transport by solving the convection-dispersion equation. SWMS-3D includes a water extraction term in the Richard's equation which can be used to simulate water uptake by roots. The solute transport model also includes an extraction term for nutrient uptake by roots. The water uptake by roots by dividing the potential transpiration equally over the root length of the root system was calculated. The nutrient uptake by the roots using Michealis-Menten kinetics was calculated. Thus nutrient uptake becomes a function of the nutrient concentration in the profile, while nutrient concentrations in the profile depends on the uptake. A predictor corrected approach to solving this mutual dependency was used, where the initial prediction was made using forward Euler and the final result was calculated using a backward Euler. Stability of the results was checked by checking the nutrient balance of the whole system which stayed within 1% accuracy.

In order to link SimRoot to SWMS-3D, the root nodes were matched, which are spaced 0.5-1 cm apart, to the nodes of the 1 cm cubed finite element grid (f.e.m), which was used by SWMS. To do so, all of the f.e.m. nodes within a distance of √3, i.e. the diagonal length of one finite element were matched, to the root nodes. The uptake of the root nodes were divided by the nearby finite element nodes using a weighing factor which was the relative inverse distance to the power of six. The average concentration at the root node surface by averaging the nutrient concentration of the nearby nodes using the same weighing factor. Thus, a strong preference was given to the nearest node in an effort to avoid artificially increasing the domain of soil exploration. Using only the nearest node, however, causes numerical instabilities and artificial effects on root competition. A higher resolution finite element grid would resolve some of these issues, however, a higher resolution grid increases memory usage to the power of three and computational time even more, as not only the number of nodes increases but also smaller time steps are required for solving the transport equations. A simple, one pool, mineralization model as described by Yang and Janssen (2000) was added and run for each finite element node. The mineralization parameters were varied along the vertical dimension only but allowed three dimensional variation in soil water content to influence mineralization. There were no direct interactions between the plant model and the mineralization model, but indirect effects of local drying of the rhizosphere and changes in mineral N content were allowed to effect mineralization and nutrient uptake rates respectively.

It was assumed that RCA development starts behind the cell elongation zone of a root and increases relatively rapidly over time until a maximum is reached. Thus, the highest RCA formation can be found close to the base of the root.

In non-stressed plants, higher RCA formations have been measured in thicker root classes, even when measured relatively to their cortical area. Brace roots may be an exception to the rule, as they were thick but formed little RCA.

There is significant genotypic variation in the amount RCA formation. There is also some genetic variation in the distribution of RCA within the root system. However, these differences are smaller. An empirical approach to simulating the distribution of RCA over the root system by was adopted using data of Burton (2010) for a high (w64a) and low RCA (H99) genotype. The difference between basal and middle parts of the root system was not simulated.

W64a and H99 are two inbred lines of maize (Zea mays L.) differing in RCA production, with w64a forming about three times as much RCA. These inbred lines also differ in steepness of the nodal root system with H99 being 15 degrees steeper than w64a. H99 has thicker major axes with 1.5 times higher lateral branching frequency. The laterals, however, stay short in both inbred lines. H99 has larger seed (0.28 g) than w64a (0.2 g) but lower seed P concentration, 0.36%, instead of 0.48%.

A pioneer hybrid was included in the simulations that were found to be high in RCA formation. This hybrid is much more vigorous than the inbred lines. It produces more shoot biomass, has longer lateral roots and thereby a larger root system. The thickness of the roots is in intermediate to that of H99 and w64a.

RCA formation releases resources for utilization elsewhere in the plant. The benefit of RCA depends strongly on the utilization of those resources.

RCA formation effects the size of the root system and thereby the depth of the root system. The nutrient availability was varied with depth with higher nutrient availability in the top soil. Nitrate leaching was varied by simulating two different soils, a loamy sand and a silt loam, and three different precipitation regimes, 62, 124 and 248 mm in 40 days. 124 mm in 40 days corresponds to the rainfall in Rock Springs, Pa., during the first 40 days of the 2009 growth season.

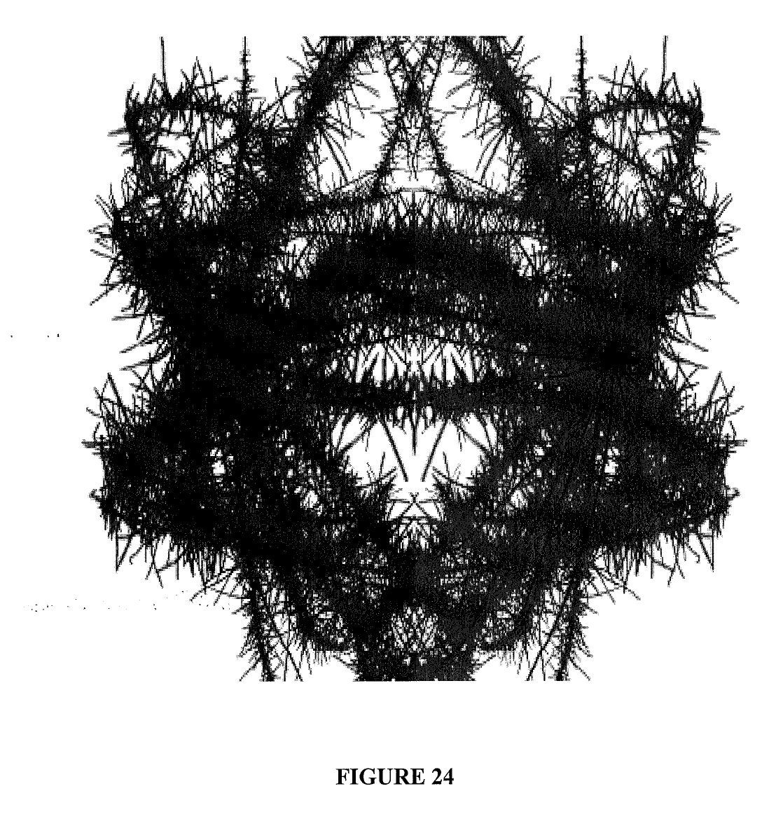

The first 40 days of growth of a single plant was a representative individual of a uniform, mono-culture, plant community with a row spacing of 60 cm and a within-row spacing of 26 cm. Aboveground competition was included by including a shading function. Belowground, realistic root density was simulated by mirroring the roots at mid-distance between the simulated and the hypothetical neighbouring plants (FIG. 24). Root competition was a result of depletion of nutrients of neighbouring roots. Root competition in the Barber-Cushman model was implemented.

In 2009, weekly soil samples were collected from the top six 10 cm soil layers in two maize fields in which RILs of the OHW population were growing. These fields were either fertilized or non-fertilized with ammonium nitrate. This verified simulated concentrations against the measured concentrations for both high and low nitrogen scenarios using a total of 60 points.

The Barber-Cushman model, a one dimension radial model was used. This approach worked well for phosphorus uptake. Phosphorus is an immobile nutrient and as a result the depletion zones around the roots are less than 5 mm. Using the Barber-Cushman model, it is easy to simulate these depletion zones at sub mm resolution. Simulating the whole soil domain at sub mm resolution, however, would require excessive computer memory and computational time. The drawback of using the Barber-Cushman model for more mobile nutrients is that, as the depletion zones become larger, interroot competition becomes more important. The Barber-Cushman model can only simulate the effect of competition one-dimensionally. Furthermore, leaching of nitrate in the soil profile cannot be simulated with the Barber-Cushman module. Therefore, the SWMS3D module was used, which is a 3D finite element model, to simulate nitrate transport in the soil.

Potassium has intermediate mobility in soils. Potassium depletion zones around roots are typically in the one cm range. The Barber-Cushman module and the SWMS3D module were used to simulate the benefit of RCA on low potassium soils to test the influence of model choice on our results.

SimRoot uses a random number generator to simulate distributions for growth rates, direction and branching frequency. The non-linearity of many processes causes this variation to be functional for the plant. For example, consider two plants with the same total lateral root length, wherein one with laterals varies in length, and one with laterals equals in length. The results indicate that the plant with varying length laterals will outperform the plant with equal length laterals.

In total more than a 1000 runs were run on the PennState clusters LIONXI and LIONXJ. The following parameters were varied:
1) the formation of RCA,
2) the functional benefit of RCA, either I) a relocation benefit or II) a respiration benefit, or III) both
3) the availability of nitrate, phosphorus and potassium in the soil,
4) the soil type: sandy and clay loam,
5) the precipitation: 62, 124, 248 mm in 40 days
6) the genotypes: three different genotypes H99, w64a and 36H56 7) our ‘normal’ genotype which is based on average numbers of multiple experiments, with and without RCA formation in the lateral roots.

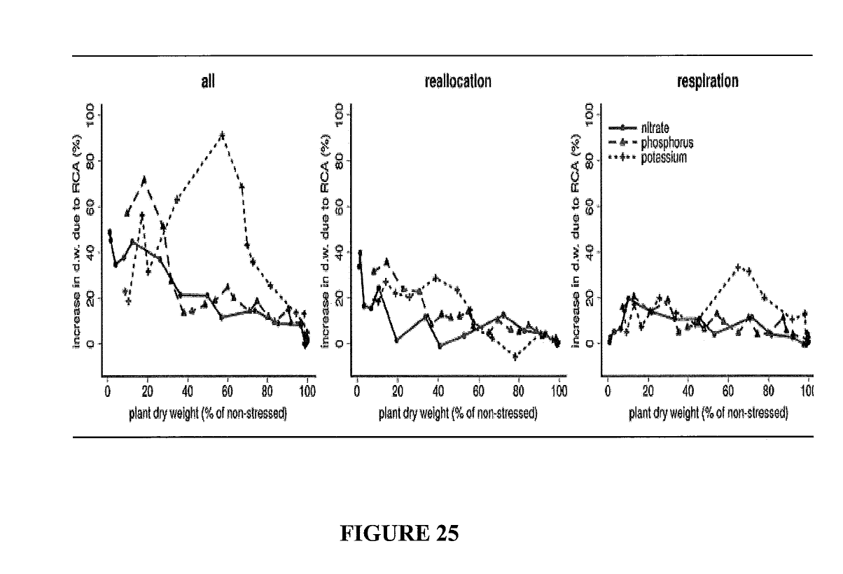

RCA had a positive effect on plant growth under all three macronutrient deficiencies (FIG. 25). The benefit of RCA depended on the intensity of the nutrient deficiency and the nutrient involved. At low to medium deficiencies, RCA formation had the greatest benefit when potassium was limiting compared to nitrogen and phosphorus, while in strongly deficient plants, RCA formation had the greatest benefit when phosphorus was limiting. The benefit of RCA generally increased with increasing nutrient deficiency, but the benefit of RCA peaked at medium deficiency levels when potassium was limiting. Plants that form RCA benefited most from reallocating nutrients and to a lesser extend from a reduction in respiration. However when potassium was limiting growth, a reduction in respiration became the more important function of RCA formation.

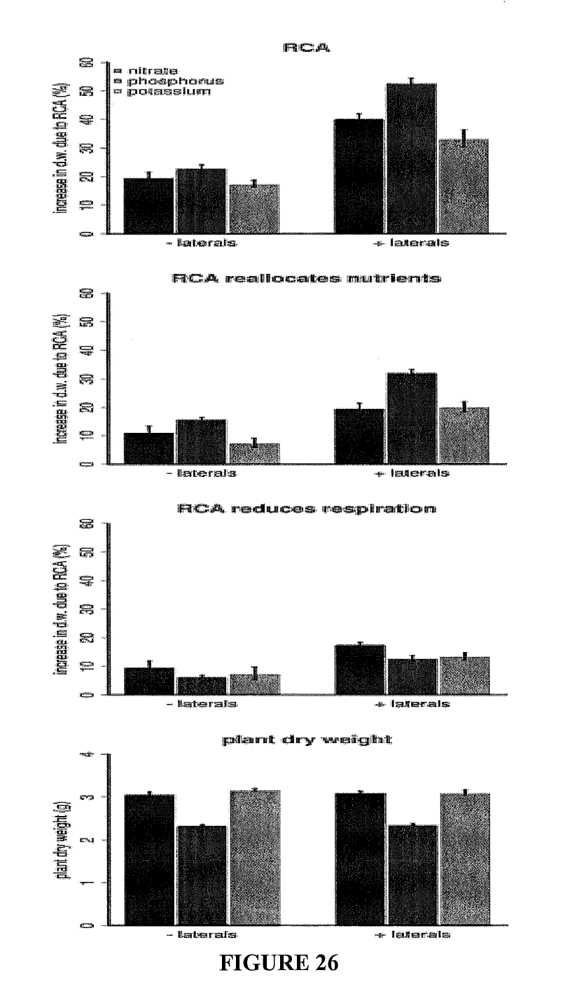

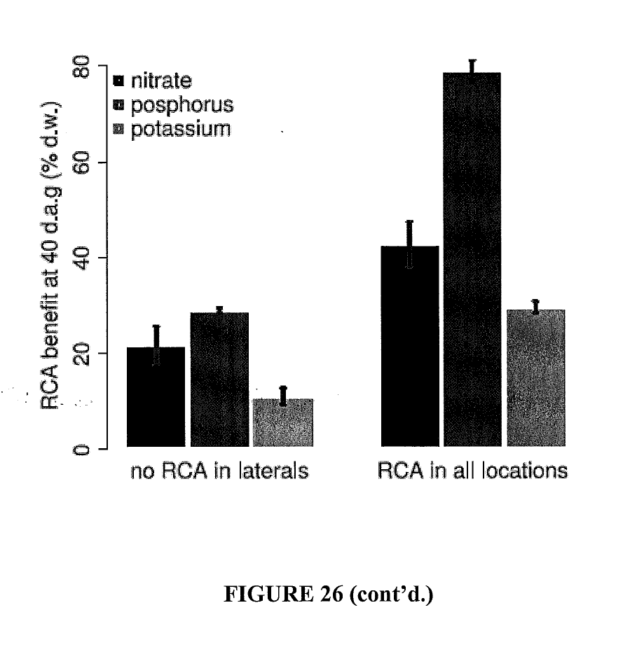

The model predicted much larger benefits of RCA in plants that form RCA in lateral roots (FIG. 26). This benefit of RCA formation in lateral roots was stronger when phosphorus was limiting growth. The reallocation function of RCA was more affected by RCA formation in the laterals than the respiration function.

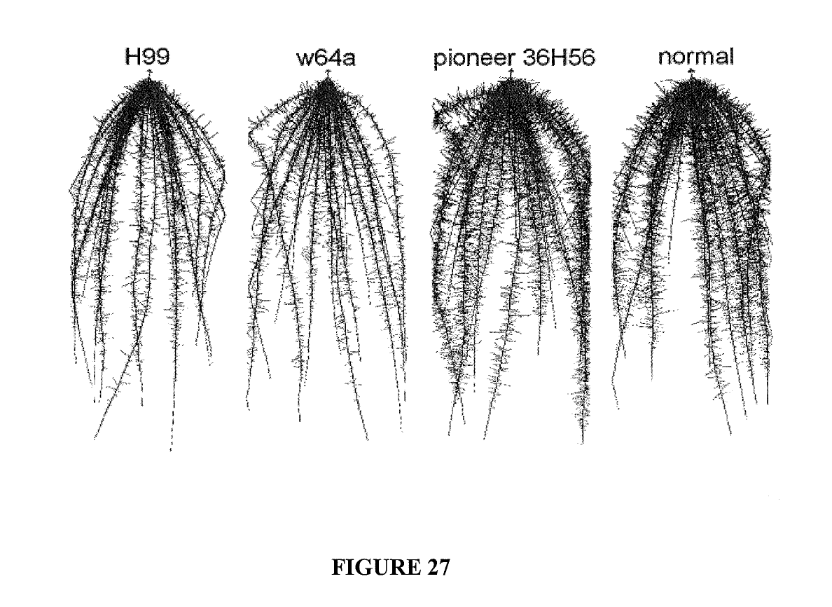

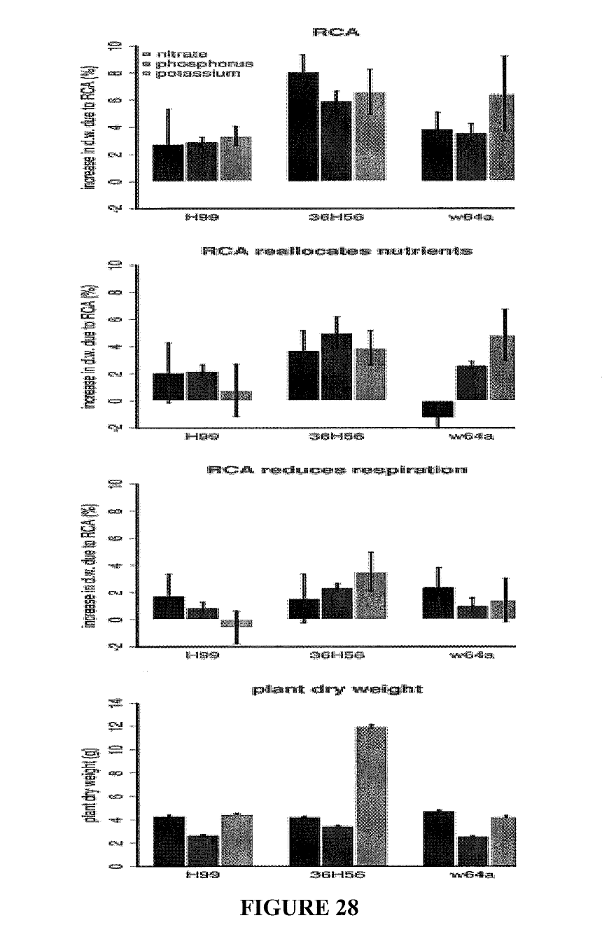

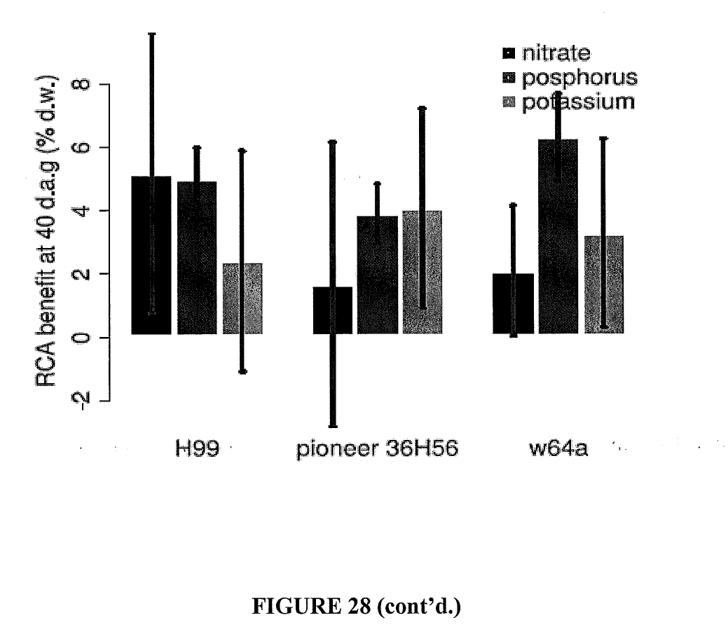

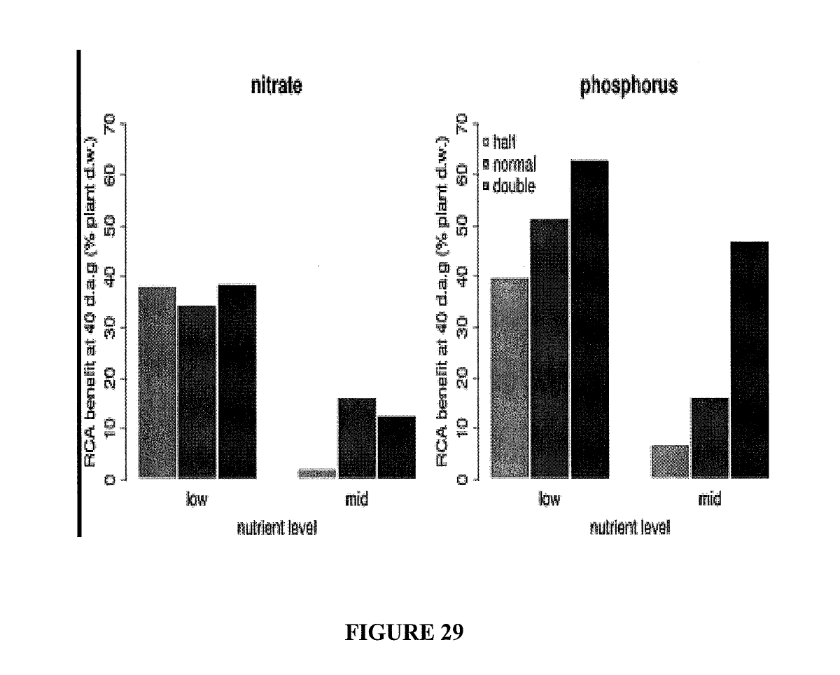

The root architecture of three genotypes was simulated (FIG. 27). These genotypes differ in RCA formation, lateral branching, steepness of the root system and growth potential. The distribution of RCA formation for these three genotypes was simulated. The benefit of RCA formation in these genotypes is low, less than 10 percent (FIG. 28). RCA formation increased growth in all genotypes, especially in the higher RCA genotypes, w64a and 36H56.

The virtual reference plant was used (“normal”, FIG. 27) form equal amounts of RCA in all roots including laterals, to simulate the benefit or RCA formation under nitrate and phosphorus deficiency, given different lateral branching densities. In soils with moderate N availability, RCA benefited plants with normal lateral branching density the most.

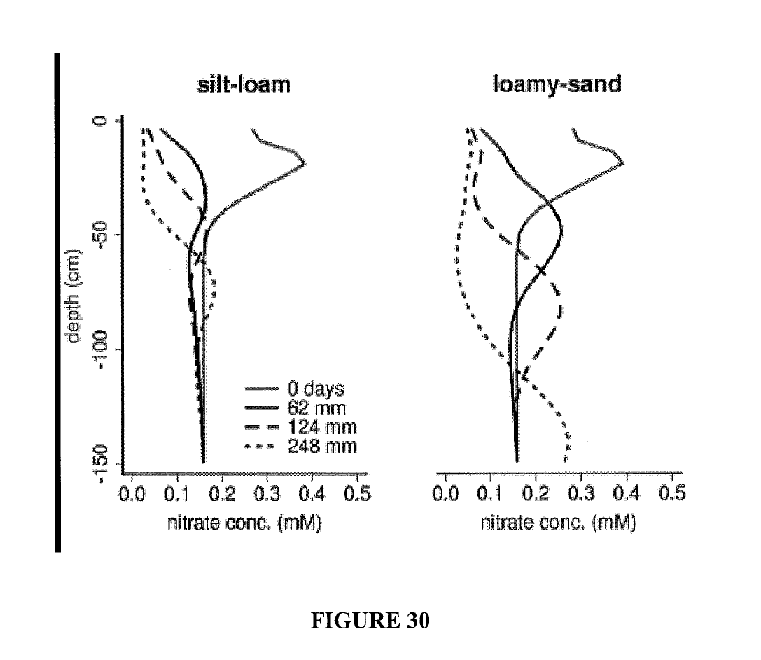

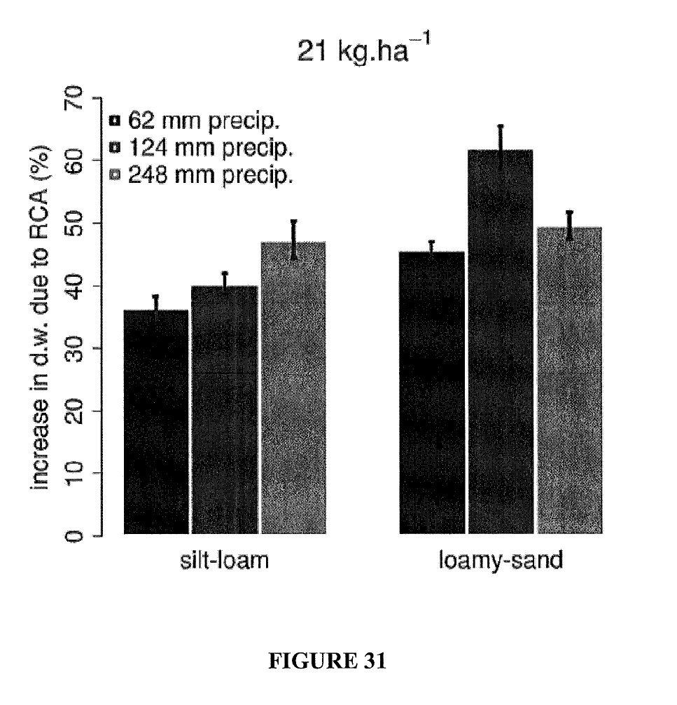

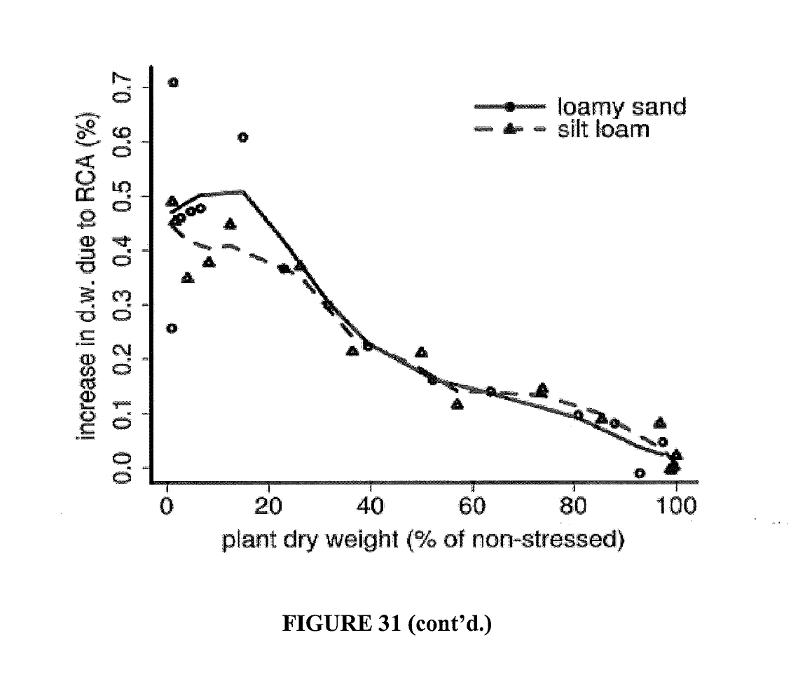

A loamy sand and silt loam soil was simulated with three levels of precipitation to vary the intensity of nitrate leaching. As expected, nitrate leaching increased with increasing precipitation (FIG. 30). This increase was greater in the loamy sand than in the silt loam. Plants benefited more from RCA formation in high leaching environments than in low leaching environments (FIG. 31). As shown in FIG. 28, variation, as depicted by the standard error bars, was introduced by simulated variation in growth rates, direction and branching frequencies of individual roots.

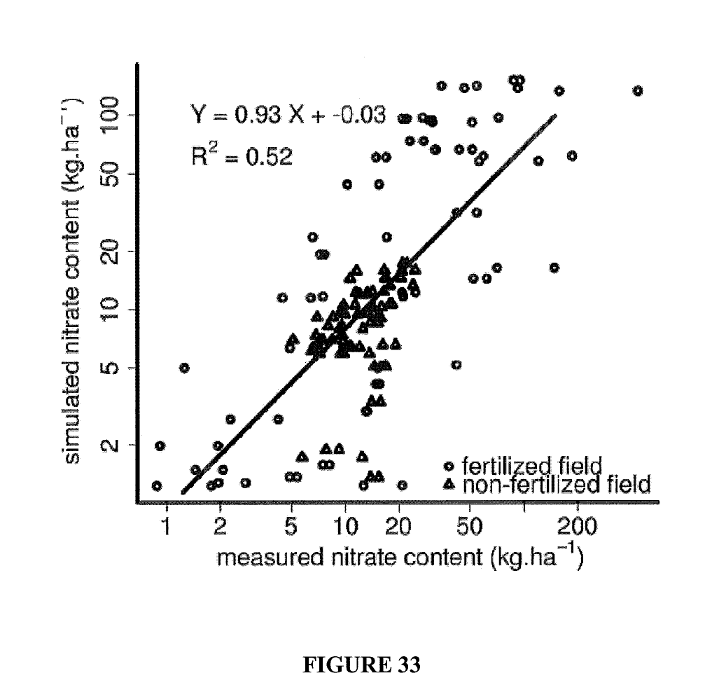

The simulated nitrate concentrations at different depths over time correlated positively (R²=0.52) with the measured nitrate concentrations (FIG. 33). The regression line had a slope of near one and an intercept of near seven and ten (FIG. 33).

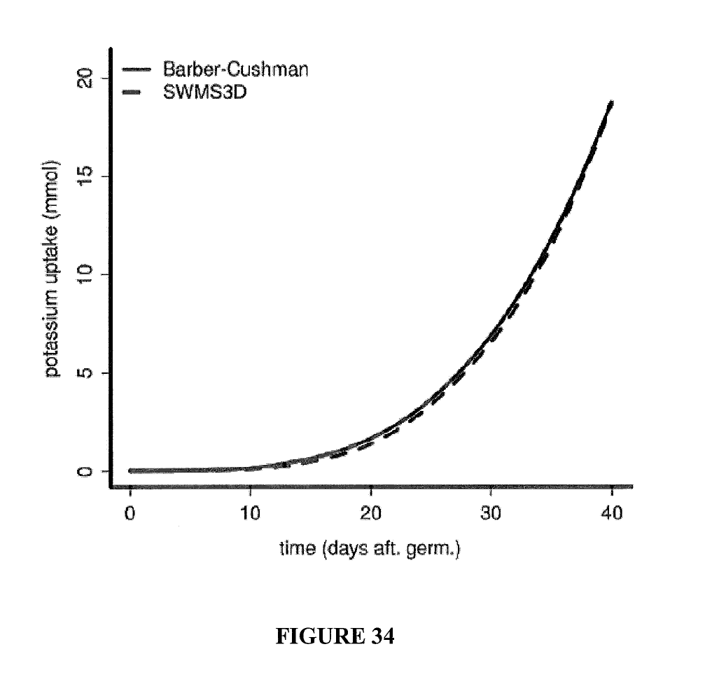

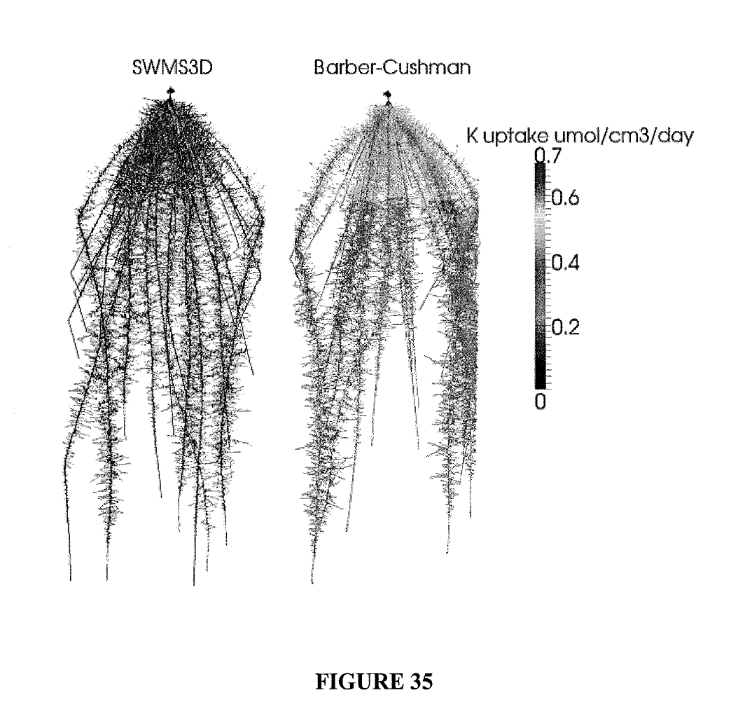

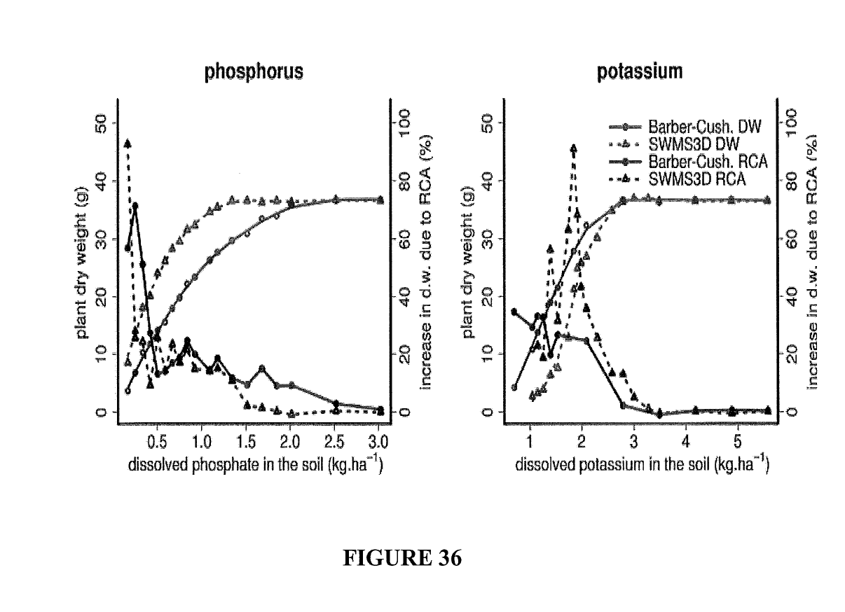

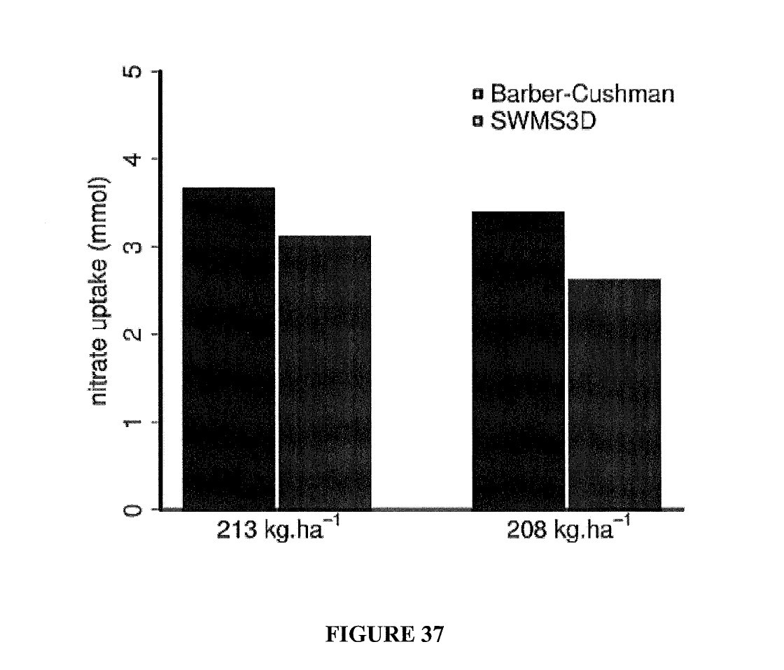

Both the Barber-Cushman and SWMS3D modules simulated very similar amounts of potassium uptake when potassium availability was high (FIG. 34). However, the spatial-temporal distribution of uptake differed strongly between the models (FIG. 35). The uptake per root class and over time varied more strongly in the SWMS3D model. The Barber-Cushman model simulated significantly higher total uptake than the SWMS3D model when potassium availability was lower which resulted in significantly more growth (FIG. 36). The steeper response curve of the SWMS3D model to potassium availability (FIG. 36) caused aerenchyma to be more beneficial in the SWMS3D model (FIG. 36). In accordance to our hypothesis, the SWMS3D model simulated greater uptake for phosphorus while the Barber-Cushman module simulated greater uptake for nitrate (FIG. 37).

Discussion

RCA forms in response to a deficiency of nitrogen, phosphorus and sulphur. The simulation results demonstrate, in part, that RCA formation substantially benefits plants experiencing deficiencies of nitrogen and phosphorus, and suggests that RCA be beneficial under potassium deficiency as well (FIG. 25).

Reallocation of nutrients is predicted by the model to be the more important function of RCA formation in nitrogen and phosphorus deficient plants (FIG. 25). The photosynthetic efficiency of leaf area by potassium deficiency, and the lack of an adaptive change in shoot-root allocation in potassium deficient plants causes root growth to become more carbon limited than in phosphorus or nitrogen deficient plants. Hence, a reduction in respiration mitigates this carbon limitation in potassium deficient plants.

According to the simulation results, RCA formation in lateral roots would benefit nutrient deficient plants (FIG. 26). These results suggest that lateral roots, despite their fineness, should not be ignored. The respiration function of RCA is less important than the reallocation function in lateral roots. While reallocation of nutrients from RCA benefits the plant as RCA is formed, a reduction in root respiration benefits the plant over the life time of the plant. Thus, relative importance of these functions changes over time. The younger age of later roots in comparison to the major axis from which they branch explains the relative smaller respiration benefit from RCA formation in lateral roots. Fine root turnover will increase the relative age difference and simultaneously decrease the importance of RCA formation in lateral roots. This suggests that carbon and nutrient requirements for growth of lateral roots may be more important costs than the maintenance respiration of lateral roots.

The RCA distribution was simulated in three different genotypes (FIG. 27) and all three genotypes benefited from RCA formation when grown on low nitrogen, phosphorus or potassium soils (FIG. 28).

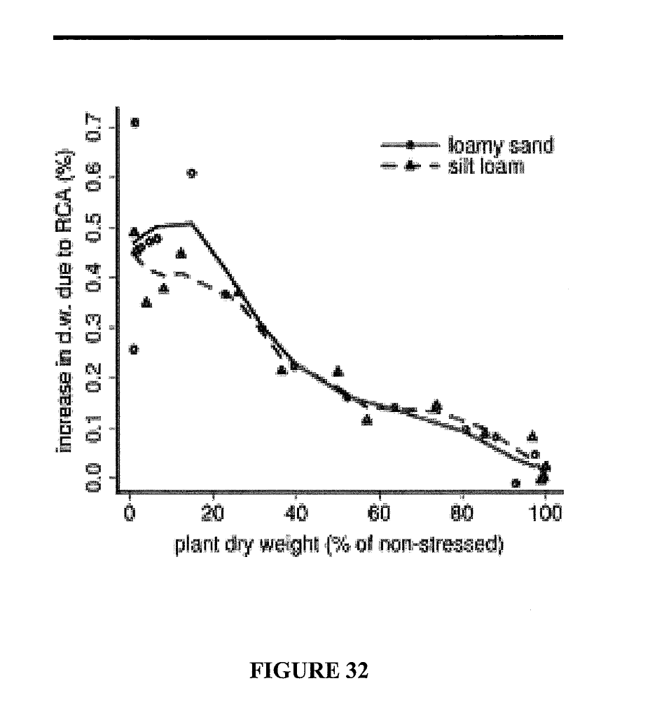

Nitrate leached faster in the loamy sand than in the silt loam soil, and increased with greater precipitation (FIG. 31). The benefit of RCA was greater in higher leaching environments (FIG. 32). The greater benefit of RCA in sandy soils can be explained by the increased nutrient deficiency in these soils (FIG. 34) which is caused by the increased nitrate leaching.

### CONCLUSIONS

RCA is a trait that provides improved plant growth under nitrogen, phosphorus and potassium deficiency, and can be part of a breeding strategy for maize production, as well as other plants, to increase capture of soil resources such as nitrogen.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

The invention claimed is:

1. A method of increasing capture of a soil resource from soil by a plant comprising:
(a) determining a first amount of root cortical aerenchyma in a first plant;
(b) determining a second amount of root cortical aerenchyma in a second plant;
(c) identifying an improved soil resource capturing plant by comparing the first amount of root cortical aerenchyma in the first plant and the second amount of root cortical aerenchyma in the second plant; and
(d) sexually reproducing or asexually reproducing the improved soil resource capturing plant, thereby forming a progeny of the improved soil resource capturing plant.

2. The method according to claim 1, further comprising planting the progeny.

3. The method according to claim 1, wherein the soil resource is a nutrient selected from the group consisting of phosphorus, potassium, and nitrogen.

4. The method according to claim 1, wherein the soil resource is nitrogen.

5. The method according to claim 1, wherein the reproducing step is cross-pollinating the improved soil resource capturing plant with the first plant or a third plant.

6. The method according to claim 1, wherein the improved soil resource capturing plant is asexually reproduced by self-pollination.

7. The method according to claim 2, wherein the progeny is a seed.

8. A method of reducing an amount of fertilizer used on a crop comprising:
(a) determining a first amount of root cortical aerenchyma in a first plant;
(b) determining a second amount of root cortical aerenchyma in a second plant;
(c) identifying an improved soil resource capturing plant by comparing the first amount of root cortical aerenchyma in the first plant and the second amount of root cortical aerenchyma in the second plant; and
(d) planting the crop comprising seeds from the improved soil resource capturing plant or seeds from a progeny of the improved soil resource capturing plant.

9. The method according to claim 8, wherein the fertilizer comprises a nutrient selected from the group consisting of: phosphorus, potassium, and nitrogen.

10. The method according to claim 8, further comprising crossing the improved soil resource capturing plant with the first plant or a third plant, thereby forming the progeny of the improved soil resource capturing plant, wherein the crop comprises seeds from the progeny of the improved soil resource capturing plant.

11. The method according to claim 9, wherein the nutrient is nitrogen.

12. A method for determining a plant having an improved ability to capture a soil resource comprising:
(a) determining a first amount of root cortical aerenchyma in a first plant;
(b) determining a second amount of root cortical aerenchyma in a second plant; and
(c) identifying the plant by comparing the first amount of root cortical aerenchyma in the first plant and the second amount of root cortical aerenchyma in the second plant.

13. The method according to claim 12, further comprising planting the plant in soil having a low amount of the soil resource.

14. The method according to claim 12, further comprising sexually or asexually reproducing the plant, thereby forming a progeny of the plant.

15. The method according to claim 14, further comprising planting the progeny of the plant in soil having a low amount of the soil resource.

16. The method according to claim 12, wherein the soil resource is a nutrient selected from the group consisting of: phosphorus, potassium, and nitrogen.

17. The method according to claim 12, wherein the first amount of root cortical aerenchyma is determined by measuring a percentage of root cortical aerenchyma present in a first cross-section area of a first root from the first plant.

18. The method according to claim 12, further comprising growing the second plant under a set of conditions and for a period of time substantially equal to the set of conditions and the period of time used to grown the first plant.
